(19) United States
(12) Patent Application Publication
(10) Pub. No.: US 2005/0220882 A1
(43) Pub. Date: Oct. 6, 2005

(54) Materials for medical implants and occlusive devices

(76) Inventors: Wilson Pritchard, Memphis, TN (US); Cedric Flowers, Bartlett, TN (US); Tony Prescott, Arlington, TN (US); Rick Mendius, Collierville, TN (US); Clive Hallam, Memphis, TN (US)

Correspondence Address: PATTERSON, THUENTE, SKAAR & CHRISTENSEN, P.A., 4800 IDS CENTER, 80 SOUTH 8TH STREET, MINNEAPOLIS, MN, 55402-2100, US

(21) Appl. No.: 11/071,866

(22) Filed: Mar. 3, 2005

Related U.S. Application Data
(60) Provisional application No. 60/550,132, filed on Mar. 4, 2004.
(60) Provisional application No. 60/557,368, filed on Mar. 29, 2004.
(60) Provisional application No. 60/564,858, filed on Apr. 23, 2004.
(60) Provisional application No. 60/637,569, filed on Dec. 20, 2004.

(51) Int. Cl. A61K 31/715; A61K 9/48; A61K 33/38

(52) U.S. Cl. 424/488; 424/618; 514/54

(57) ABSTRACT

An embodiment is a swellable medical device that swells after introduction into a patient to occlude a lumen or void in a patient. The device may be anisotropically swellable so that it swells unequally in some dimensions to create an improved fit of the device into the patient. Anisotropically swellable materials are also described. Further, materials and methods for removing a biocompatible hydrogel from a patient by a metal-catalyzed oxidative-reductive reaction are described. Other embodiments are directed to devices that are shrinkable, dissolvable, or otherwise removable by exposure to deionized water or hypertonic solutions. Certain other embodiments are materials and methods for making and using chelation-resistant materials crosslinked by insoluble metal salts.

### RELATED APPLICATIONS

This application claims priority to U.S. Patent Application Ser. Nos. 60/550,132 filed Mar. 4, 2004, 60/557,368 filed Mar. 29, 2004, 60/564,858 filed Apr. 23, 2004, and 60/637,569 filed Dec. 20, 2004, each of which are hereby incorporated by reference herein. This application is also related to U.S. patent application Ser. No. ______, entitled OCCLUSIVE BIOMEDICAL DEVICES, PUNCTUM PLUGS, AND METHODS OF USE THEREOF, filed ______.

### FIELD OF USE

The field of use is related to occlusive medical devices, and includes disclosure of medical occlusive devices such as plugs placed into a lumen or void of a patient for occluding the same.

### BACKGROUND

Occlusive medical devices are useful for a variety of applications, for example, occluding blood vessels, occluding other lumens such as fallopian tubes, filling aneurysm sacs, sealing arteries, and closing punctures. Occlusion of blood vessels may reduce blood flow to tumors, uterine fibroids, or for treatment of vascular malformations, such as arteriovenous malformations (AVMs) and arteriovenous fistulas (AVFs). Occlusive medical devices may also be adapted to stop or slow bleeding. These and other applications require suitable materials and devices that may be introduced to the application site to perform the appropriate function.

Some occlusive medical devices and materials for implantation into a patient can be exposed to chelation agents after they have been introduced into the patient. For example, plugs placed in the punctum of the eye are exposed to chelating agents in topical ophthalmic solutions. A chelation agent is an organic chemical that bonds with a free metal ion and thereby removes it from solution. Some implanted materials are susceptible to weakening and dissolution by chelation agents, so that the exposure of these materials to chelation agents causes loss of crosslinking and imparts solubility in water or body fluids.

### SUMMARY OF THE INVENTION

This application describes various materials and methods for making occlusive medical implants. Certain embodiments describe chelation-resistant implantable materials, including materials that are degradable over short term, degradable over a long term, or effectively undegradable. Certain embodiments of these materials are, furthermore, triggerably degradable upon exposure to triggering agents that cause the materials to be essentially completely or partially degraded.

Occlusive medical devices can be made of swellable materials. A controlled amount of swelling can be useful to set the implant in place, but too much swelling can harm surrounding tissue. A tissue is a solid or partially solid portion of a patient's body. Tissues that surround a preexisting or created space in a body define that space, e.g., the walls of an artery define the artery lumen, and the tissue around a bolus of material injected into a muscle defines the space thereby created. In some circumstances, the implant must be firmly set into an opening in a patient so that a relatively high degree of swelling is desirable, but the high degree of swelling tends to push the implant out of the opening so that the implant is not stable. Accordingly, controllably swellable materials may be used, as described, below.

Certain other embodiments provide for materials and devices that are controllably and anisotropically swellable, meaning that the materials or devices are designed to swell more in one direction than in other directions. For example, cylindrical rods may be constructed to have a diameter that increases in response to swelling, and have a length that increases to a lesser extent, essentially does not increase, or even shrinks. Other embodiments are provided that have a combination of these and other features, e.g., chelation resistance, triggerable dissolution, long-term or short-term degradation, and anisotropic swelling.

### BRIEF DESCRIPTION OF THE DRAWINGS

FIG. 1 depicts the molecular structure of Gellan.

FIG. 2 depicts the molecular structure of cellulose.

FIG. 3 depicts an anisotropically swollen device (a plug) before (right hand side) and after (left hand side) swelling, with the striped lines being a scale; the swelling has caused an increase in diameter and a decrease in length.

FIG. 4 depicts Schirmer data collected for another embodiment of an occlusive device.

### DETAILED DESCRIPTION OF THE DRAWINGS

Various materials and methods for making improved occlusive devices are described herein. Certain embodiments are directed to occlusive devices that are swellable, anisotropically swellable, chelation resistant, controllably degradable, triggerably degradable, and gellable by physiological fluids. Embodiments include swellable devices that expand in response to physiological fluid. And other embodiments are anisotropically swellable devices that are swellable in a lumen or void to expand radially, but not longitudinally, whereby the device fits securely without being dislodged by longitudinal extension. And certain devices described herein are degradable at a predetermined rate by virtue of materials that are incorporated into their structure. Also disclosed are devices made of materials that are degradable upon exposure to a triggering substance that causes degradation. Other devices and materials are also disclosed, including plugs made from expandable foam and compositions that gel upon exposure to physiological fluids.

Resistance to chelation may be advantageous for occlusive devices that are exposed to chelating agents. Accordingly, some embodiments describe chelation-resistant implantable materials, including materials that are degradable over short term, degradable over a long term, or effectively undegradable.

While some conditions are best treated with permanent or nondegradable occlusive devices, the use of temporary occlusive devices can be beneficial in some situations. Examples of permanent and temporary occlusive devices are described, below. Various materials and methods of processing these materials are also described.

Gellan, Depolymerized Gellan, and Related Polysaccharides for Biomedical Uses

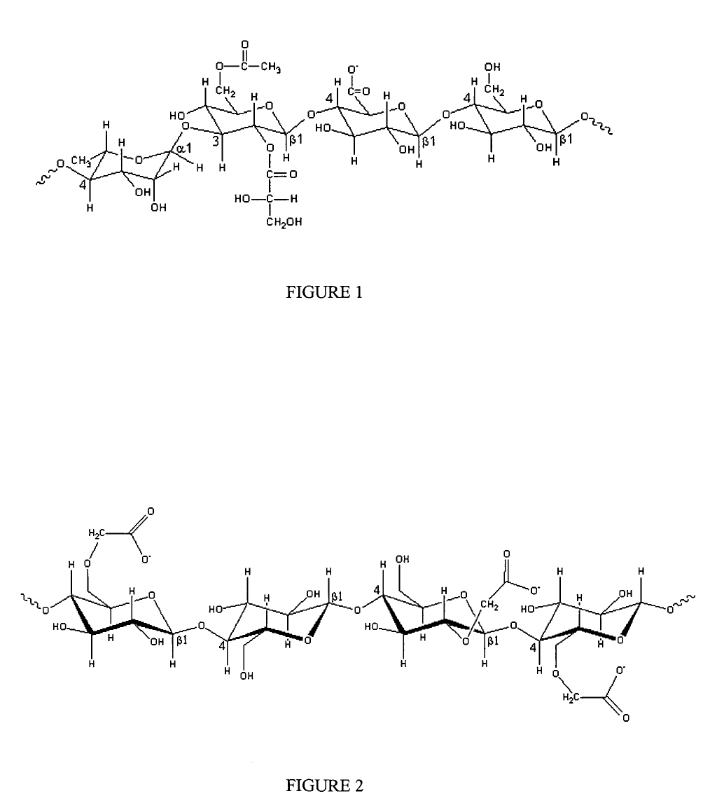

Biomedical devices may be made using gellan, depolymerized gellan, and related polysaccharides. As set forth in greater detail in U.S. Patent Application Ser. No. 60/557,368, gellan gum is a polysaccharide, and is prepared commercially as a bacterial exopolysaccharide using fermentation, e.g., from Sphingomonas elodea (previously called Pseudomonas elodea). FIG. 1 shows the structure of a form of gellan. The properties of a gellan-based material depend, in part, on the degree of gellan's acylation and the ions present. If left acylated, gellan tends to form soft, elastic, transparent and flexible gels. When de-acylated it forms hard, relatively non-elastic brittle gels. A gellan gum solution may hold particles in suspension without significantly increasing the solution's viscosity. A gel sol transition occurs at about 50° C. dependent on concentration. Thermoreversible gels form on cooling in the presence of cations even at low (0.1% w/w) to very low (0.005% w/w) concentrations of gellan. Its ability to form gels even in the presence of monovalent cations alone makes it unique compared to other commercially available gel-forming polysaccharides. Gellan can be formulated at concentrations and conditions so that it gels in response to exposure to physiological conditions.

Gellan, as received from a typical supplier, e.g., CPKelco, contains metal impurities including calcium and magnesium. Without their removal, making of concentrated gellan solutions can be very difficult or impossible (if room temperature processing is desired). In general it is found that gellan purified to its sodium or ammonium salt is soluble in water at room temperature. Solubility at room temperature is limited to low (<5%) concentrations of gellan.

Gellan gum is typically used at concentrations below about 2%, but may be mixed to higher concentrations if suitable steps are taken such as purification to monovalent salts, variation of solvents, or neutralization of charged groups prior to use of an organic solvent. Gelation of concentrated aqueous or aqueous/organic solutions without additional counterions allows creation of concentrated fluid gels. Fluid gels have excellent suspension properties and can hold particles at very high loadings with no increase in viscosity. They are normally made, for example, from 0.4-0.6% gellan plus a counterion. After heating, the mixture is allowed to cool under vigorous stirring. Use of higher concentrations of monovalent salts without additional counterions allows for much more concentrated fluid gels to be made and is particularly useful as a drug delivery vehicle, a suspender/binder inert or bioactive ceramics and glasses, etc.

If sodium gellan is made to about a 2% solution without heating, hydration is typically attained. Room-temperature stable concentrated (10%) solutions can be obtained by evaporation of water under vacuum or ambient conditions. This solution can then be easily used to make extrusions at room temperature. Highly concentrated gellan solutions stable at room temperature could be injected and used for soft tissue augmentation, drug delivery, etc. As the gel hydrates, it also expands (up to 500% or more depending on the concentration of gellan and the strength of the ionic bonds). After hydration, the gellan becomes pliable and malleable to conform to the inside of the volume that constrains it (assuming the volume is less than or equal to the physical size of the gel in its hydrated state).

Gellan has a long history of clinical use in humans that spans 15 years. It has been studied as a drug delivery material because of its in situ gelling properties. It has also been studied as a time release material for drug delivery for its controllable and predictable dissolution properties (as a gel) in contact with mucosal membrane (analogous to the punctum) in vivo, and for insulin delivery in vivo. And gellan has been studied for both its gelling properties and dissolution rate. Several studies have been completed dealing with the safety of gellan for use in the eye. And more specifically, numerous studies involving gellan as a safe and efficacious delivery vehicle for TIMOLOL (antiglaucomatous medication) have been completed.

Polysaccharides closely related to gellan are those such as welan, S-88, S-198 or rhamsan gums; these can also be processed by the methods described herein, and can be used as substitutes for, or added to, gellan gum. Other polysaccharides related to gellan are alginate, curdlan, carboxymethylcellulose, crosscarmellose, poly(acrylic acid), xanthan, carrageenan, carboxymethyl chitosan, hydroxypropyl carboxymethyl cellulose, pectin, gum Arabic, karaya gum, psyllium seed gum, carboxymethyl guar, and mesquite gum; methods described herein can be generally adapted for use with these polysaccharides.

As described in greater detail below, some embodiments are materials and devices that resist degradation, resist chelation, and are at least partially made of gellan. Sodium gellan is unaffected by disodium EDTA, a chelating agent. Disodium EDTA can exchange its sodium ions for crosslinking ions in a given ionically-crosslinked hydrogel. Unlike many other ionic, gelling polymers such as sodium alginate, sodium gellan remains a gel in vivo. Hence removal of divalent or trivalent ions and conversion to sodium gellan does not affect the physical state of the hydrogel. Gels strong enough to be used as implantable plugs may be dense and, to that end, may be processed from at least 5% gellan gum in water or DMSO. Other concentrations include between 1% and 50%, including 5%-15%, and 15%; persons of ordinary skill in these arts will appreciate that all values and ranges within the explicit limits are contemplated. Gellan will not normally resorb or dissolve after implantation into a patient, but can be removed by exposure to salt-free water.

Swellable Materials and Devices

As a dry gel material hydrates, it typically swells to fill a space and then takes up no more water. For example, if a dry gel material is placed in thin walled flexible silicone tubing and then hydrated, the gel will swell to fill, but only slightly deform, the tubing. A hydrogel plug that incorporates an unconstrained hydrogel material will thus be more successful in swelling to achieve a secure fit. This unconstrained hydrogel material may be located at, e.g., the bottom or nose of a plug. The top end of a plug, the neck and rim, may include a strong, non-swelling material to address the issues of cutting strength and dimensional stability. For example, a nonswelling plastic may be used to cover the upper portion of a polysaccharide plug so that the polysaccharide will swell against the plastic but not further expand. The other portion of such a plug, however, will be free to swell. When a hydrogel's expansion is limited by a constraining tissue, the hydrogel exerts a force against that tissue.

Swellable means something that can be swollen in response to a fluid. Some hydrogels are swellable because they are less than fully hydrated when introduced into a patient, so that the hydrogel imbibes fluid from the patient. Such hydrogels may be, e.g., desiccated, lyophilized, or hydrated but not fully hydrated. A hydrogel that has been dehydrated to remove water is referred to herein as a hydrogel. Hydrogels do not dissolve in solution. Certain materials that are specially prepared to dissolve or otherwise break up in substantially deionized water, but not physiological solutions, are referred to herein as hydrogels since they are chemically crosslinked and do not dissipate under the conditions of their intended use prior to their intentional removal with deionized water.

Gellan, polysaccharides closely related to gellan, and other polysaccharides related to gellan may be used to make swellable occlusive devices, e.g., punctum plugs. Swelling of a polysaccharide may be, for example, between 25% and 1000% as measured in a physiological solution without restriction. Swellable plugs may be made with essentially randomly oriented polymers so that there is no preferential direction of swelling in the polysaccharide portion of the plug.

Gellan gum was acidified by washing three times with 5% citric acid in water. Resulting acidified gellan powder was subsequently rinsed with water and alcohol and allowed to dry. Acidified powder (15 grams) of gellan gum was dissolved into 100 milliliters of dimethyl sulfoxide to make a 15% solution which was subjected to a vacuum to remove air bubbles. This solution was extruded under air pressure (45-50 pounds per square inch) into 10% sodium citrate in water and allowed to incubate for 30 minutes. It was subsequently washed in 1.0% sodium chloride to remove any excess citrate ions. Extrusions were dehydrated in a graded alcohol series to 91% alcohol and either stretched to twice their original length or left unstretched. They were allowed to air dry. Prototype occlusive devices were fabricated by cutting neutralized extrusions into cylindrical pieces. Their dry dimensions were 1.524 millimeters in length and 0.254 millimeters in diameter for stretched extrusions and 0.762 millimeters for unstretched extrusions. Once placed into physiological saline and allowed to swell to their maximum extent, stretched extrusions shrank to 1.27 millimeters in length and swelled to 1.016 millimeters in diameter. This represents a 16.6% decrease in length and a 300% increase in diameter. Unstretched extrusions swelled to 2.54 millimeters in length and 1.27 millimeters in diameter. This represents a 166% increase in both length and diameter. The measurements were made using a scale marked in increments of 0.01 inches, and were then converted to metric units.

Anisotropically Swelling Materials and Devices

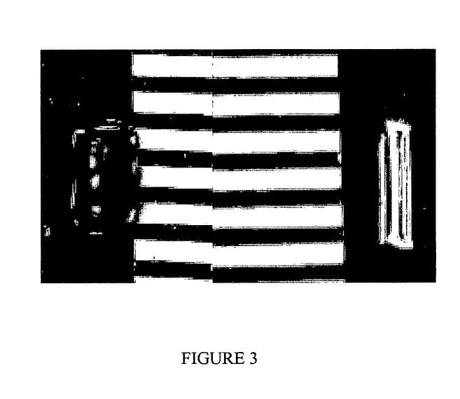

A swellable occlusive device placed into a lumen or opening can sometimes be forced out of the opening by the swelling process. Or a portion outside the opening can swell to make appropriate placement difficult. It is therefore helpful in some situations to use a device which swells only in lateral dimensions, thus effectively blocking, but not protruding from, the opening, e.g., a duct or canal. Further, the device may shrink in at least one dimension, such that a thin, cylindrical device becomes short and fat once hydrated. Punctum plugs, for example, may be made with anisotropically swelling materials. FIG. 3 depicts an example of a swellable device made of substantially parallel polysaccharides, with the striped lines indicating dimensions before and after swelling. The dimensions are actual results but are exemplary only, and may be suitably modified in light of the material used and the properties of the lumen or void that receives it.

An anisotropically swellable material does not swell equally in all directions. When unrestrained, such materials swell differentially. For example, an anisotropically swellable hydrogel may swell only in one or two directions while maintaining or diminishing in another direction. When restrained, such materials apply a greater force in the direction in which they preferentially swell. An anisotropically swellable polymer material may be prepared by aligning polymer molecules in one or more preferential directions. Polymer molecules are arranged randomly and thus tend to move apart in all directions upon hydration, and thus conventional undergo isotropic swelling (essentially the same in all directions). If polymer molecules are aligned parallel to each other, however, they move apart in only one or two dimensions, as they are (ideally) already fully extended in a third. Upon hydration, molecularly aligned hydrogels would demonstrate anisotropic expansion. Some anisotropic materials comprise polymers that are substantially parallel to each other in their molecular orientation, with the material having enough such polymers so that its macroscopic swelling properties are affected. Hydration, in its strictest sense, refers to a process involving water, but other liquids can also serve to accomplish the swelling of polymers, and such processes are contemplated herein. In some embodiments, hydrogels are fabricated by crosslinking of water-soluble polymers so that the crosslinking is only extensive enough to insolublize the material in water. Upon hydration, the oriented polymer molecules are forced apart, held together only by crosslinks.

Anisotropically swellable materials may be prepared as described, below, or as already described, e.g., as in U.S. Patent Application Ser. No. 60/557,368 or 60/637,569, and made into a device for occluding a lumen or void. The device may include an introducible portion that is introducible into the lumen or void, wherein at least a part of the introducible portion comprises an anisotropically swellable material that anisotropically swells in vitro in a physiological saline solution when not subjected to constraining forces. A physiological saline refers to a solution having a pH in a physiological range, e.g., in a range of about 7.0 to about 7.4 and an osmolarity in a physiological range, e.g., between about 300 and about 330 milliOsmoles. Phosphate buffering systems, and others, are known for making physiological salines.

A material may be tested for anisotropic swelling by measuring a sample's dimensions before and after exposure to a large excess of physiological saline, with final measurements being conducted when the swelling of the material has essentially ceased. In the case of a plug, the plug's dimensions could be measured in a state that is equivalent to its conditions immediately prior to insertion into a patient, and after exposure to the physiological saline in vitro. Unless stated otherwise, reported swelling measurements are made at room temperature (about 20° C.), but degradation in physiological saline is discussed in the context of physiological temperatures (37° C.).

Use of anisotropic hydrogels as materials for punctal occlusion solves a problem with many devices. The size of the punctal opening varies among patients; therefore the punctum must be measured, and a properly sized plug inserted. Devices made from anisotropic hydrogels, however, require neither measuring punctal size nor keeping of an inventory of many differently sized punctum plugs. Proper dimensions necessary for punctal occlusion are achieved through hydration of the device. For example, the device will swell radially until it has expanded sufficiently to occlude the nasolacrimal passage but will otherwise change its other dimensions in a controlled manner.

An anisotropically swellable occlusive device may include a volume, a first length and a second length perpendicular to the first length, wherein exposure to physiological fluid causes the volume to increase, the first length to undergo a first percentage increase and the second length to undergo a second percentage increase that is less than the first percentage increase for the first length. Examples of such increases, for the first or the second percentage increase, include at least about 25%, at least about 100%, at least 300%, and between about 10% and about 500%; persons of ordinary skill in these arts will immediately appreciate that all ranges and values within these explicitly set forth ranges are contemplated. Further, the second percentage increase may be, e.g., less than 100%, less than 50%, or less than 0% (i.e., shrinking), and between −50% (i.e., shrinking by one-half) and 100%; persons of ordinary skill in these arts will immediately appreciate that all ranges and values within these explicitly set forth ranges are contemplated.

Another embodiment is a device for occluding a lumen or void, the device comprising an introducible portion that is introducible into the nasolacrimal passage to at least partially block movement of a fluid through the passage, wherein at least a part of the introducible portion comprises a length and a swellable material that swells after introduction into the nasolacrimal passage to essentially occlude the passage while the swelling causes the length to increase by less than about 10%, 25%, or 0%.

In general, an anisotropically swellable occlusive device may be made from suitable polymers aligned in a predominantly parallel orientation relative to each other. Aligning the polymers may comprise at least one technique chosen from the group consisting of spin coating, spray coating, stretching, unidirectional freezing, extrusion from liquid crystalline solution, ordered convection, and stretching plus drying of an extrusion. A molecularly oriented occlusive device of cylindrical shape can be made in these ways, but the simplest and preferred method is usually by stretching and drying of an extrusion. In certain embodiments, aligning the polymers may comprise stretching the material and soaking the material in a fluid comprising a mineral acid, an organic acid or salts of monovalent cations before stretching the material. Aligning the polymers may comprise acidification of an anionic polymers or conversion to salts of monovalent cations before dissolution in organic solvents. Acidification is preferred as it allows for higher polymer concentrations in organic solvents such as DMSO. Examples of materials include sodium gellan, carboxymethylcellulose sodium, calcium alginate, and calcium gellan.

Monofilaments of a hydrogel material may be made, e.g., by extrusion and subsequent stretching to at least 1.5-2 times their original length. Upon drying, they can be cut into small cylinders for easy insertion into a duct or canal. For occlusion of the lachrymal system, these devices are typically 1.5-2 mm in length and 0.3-0.4 mm in diameter. An anisotropic hydrogel material of these dimensions may shrink in length to 1-1.5 mm and will expand laterally to a diameter of 1-1.5 mm. Persons of ordinary skill in these arts will immediately recognize that the embodiments are not limited to these particular dimensions. The dimensions and swelling characteristics of the device may be adapted for use with the contemplated lumen or void.

Stretching is preferably done after soaking of a material set forth herein, e.g., sodium gellan, carboxymethylcellulose sodium, calcium alginate or calcium gellan, in either a mineral acid, organic acid, or salts of monovalent cations. Acid removes cross linking divalent or multivalent cations and makes stretching far easier. Conversion of the polymer to a salt of monovalent cations also eliminates ionic crosslinks, making stretching easier. Strength is relatively unaffected. When making a solution for extrusion, the method of acidification depends upon the polymer and the extrusion solvent to be used. If DMSO is to be used as the solvent for an extrusion bath, it is normally preferable to acidify anionic polymers before dissolution in DMSO. In this case one can use acidified water as a coagulation bath. If water is the solvent in an extrusion solution, it is preferable to extrude into aqueous solutions of organic or metal salts before removing them by acidification. It has been found that, at least with alginate, acid coagulation baths produce weak acid gels which can be difficult to stretch.

Normally, orienting of ionically crosslinked high guluronic acid alginate, carboxymethylcellulose, and gellan is difficult and little anisotropy is achieved. Tight binding of divalent or trivalent cations results in decreased molecular mobility and is probably the main cause of poor orientation. Removal of gelling cations, however, makes the hydrogels much more plastic, so long as they do not become freely soluble in water. Therefore it is preferred that polymer carboxyl groups be acidified (protonated) or converted to alkali metal, tetramethylammonium, tetrabutylammonium, or ammonium salts in order to facilitate stretching and orientation.

Once an extrusion devoid of ionic crosslinking has been stretched, it is necessary to neutralize acid groups or re-crosslink with metal or organic salts. This can be accomplished either in aqueous solutions or water/alcohol solutions (usually 50-70% alcohol in water). Should aqueous solutions be used, it is necessary to have highly concentrated salt—usually saturated or supersaturated—to prevent swelling and disruption of orientation. If water/alcohol solutions are used, swelling is also greatly reduced, but one must use salts soluble in alcohol. This method can be used to fabricate stretched extrusions as a mixture calcium alginate and alginic acid, at approximately an 80%:20% ratio. There will thus be less stiffness and brittleness in the final product, which should make handling easier.

An anisotropically swellable material may comprise a polysaccharide, with the polysaccharides having a substantially parallel molecular orientation relative to each other. Substantially parallel refers to a condition wherein polymers have been processed to become aligned relative to each other instead of randomly coiled. In the context of anisotropically swellable materials, an anisotropic swelling in physiological saline under non-constrained conditions is required to demonstrate substantially parallel alignment. Examples of polysaccharides include gellan, polysaccharides closely related to gellan, and polysaccharides related to gellan. The anisotropically swellable material may include an acidic polysaccharide treated with acid-catalyzed depolymerization to lower the molecular weight of the acidic polysaccharide. The anisotropically swellable material may comprise an organic or inorganic counterion or a metallic ion.

Anisotropically swellable materials were made of gellan gum. Gellan gum was acidified by washing three times with 5% citric acid in water. Resulting acidified gellan powder was subsequently rinsed with water and alcohol and allowed to dry. Acidified powder (115 grams) of gellan gum was dissolved into 100 milliliters of dimethyl sulfoxide to make a 15% solution which was subjected to a vacuum to remove air bubbles. This solution was extruded under air pressure (45-50 pounds per square inch) into 10% sodium citrate in water and allowed to incubate for 30 minutes. It was subsequently washed in 1.0% sodium chloride to remove any excess citrate ions. Extrusions were dehydrated in a graded alcohol series to 91% alcohol and subsequently stretched to twice their original length. They were allowed to air dry.

The extrusions were placed into distilled water to assess neutralization, as sodium gellan, but not acidic gellan, is very soluble in distilled water. After 10 minutes the extrusions were dissolved, indicating neutralization had been achieved.

Occlusive devices were then fabricated by cutting neutralized extrusions into cylindrical pieces. Their dry dimensions were 1.524 millimeters in length and 0.254 millimeters in diameter. Once placed into physiological saline and allowed to swell to their maximum extent, they had dimensions of 1.27 millimeters in length and 1.016 millimeters in diameter.

Another set of anisotropically swellable materials were made of alginate. In another process, sodium alginate powder (15 grams) was dissolved into 100 milliliters of distilled water to make a 15% solution which was subjected to a vacuum to remove air bubbles. This solution was extruded under air pressure (45-50 pounds per square inch) into a coagulation bath of 5% calcium chloride and left to harden for 30 minutes. Extrusions were removed and washed three times in distilled water to remove unbound salt and then acidified by washing three times in 5% citric acid. Acidified alginate extrusions were again washed in distilled water and dehydrated through a graded alcohol series to 91% alcohol. Extrusions were taken from 91% alcohol and placed on a ruler to measure extent of stretching before breakage. The extrusions were found to easily be stretched to twice their original length, indicating that significant orientation could be achieved.

Dried alginic acid extrusions were placed into 5% calcium chloride in a 70% aqueous ethanol solution and allowed to incubate for two hours at which time they were removed, washed in a 70% aqueous ethanol solution for two hours, dehydrated in 91% aqueous ethanol and dried. Dried calcium alginate solutions were cut into small cylindrical pieces to simulate occlusive devices. The small pieces, 1.524 millimeters in length and 0.1905 millimeters in diameter, were placed into 0.9% sodium chloride to assess extent of swelling. After 15 minutes the dimensions were measured to be 1.27 millimeters in length and 0.508 millimeters in diameter.

Another set of anisotropically swellable materials were made of gellan gum. Gellan gum was acidified by washing three times with 5% citric acid in water. Resulting acidified gellan powder was subsequently rinsed with water and alcohol and allowed to dry. Acidified powder (15 grams) of gellan gum was dissolved into 100 milliliters of dimethyl sulfoxide to make a 15% solution which was subjected to a vacuum to remove air bubbles. This solution was extruded under air pressure (45-50 pounds per square inch) into 10% sodium citrate in water and allowed to incubate for 30 minutes. It was subsequently washed in 1.0% sodium chloride to remove any excess citrate ions. Extrusions were dehydrated in a graded ethanol series and subsequently stretched to twice their length and allowed to air dry.

After drying, extrusions were placed into a 5% solution of calcium chloride in 70% aqueous ethanol and allowed to incubate for 2 hours. After rinsing in 70% aqueous ethanol for two hours and dehydration in 91% ethanol, extrusions were allowed to air dry. Dried calcium alginate extrusions were cut into small cylindrical pieces to simulate occlusive devices. The small pieces, 1.524 millimeters in length and 0.337 millimeters in diameter, were placed into 0.9% sodium chloride to assess extent of swelling. After 15 minutes their dimensions had changed to 1.27 millimeters in length and 0.762 millimeters in diameter.

Gellan gum was acidified by washing three times with 5% citric acid in water. Resulting acidified gellan powder was subsequently rinsed with water and alcohol and allowed to dry. Acidified powder (15 grams) of gellan gum was dissolved into 100 milliliters of dimethyl sulfoxide to make a 15% solution which was subjected to a vacuum to remove air bubbles. This solution was extruded under air pressure (45-50 pounds per square inch) into 10% sodium citrate in water and allowed to incubate for 30 minutes. It was subsequently washed in 1.0% sodium chloride to remove any excess citrate ions. Extrusions were dehydrated in a graded alcohol series to 91% alcohol and subsequently stretched to twice their original length. They were allowed to air dry.

Upon drying extrusions were placed into a saturated solution of sodium tetraborate decahydrate in 70% aqueous methanol. Incubation in this medium lasted for two hours, followed by a two hour rinse in 70% methanol and 100% methanol. After the final wash, extrusions were air dried. Dried, borate-esterified sodium gellan extrusions were cut into small cylindrical pieces to simulate occlusive devices. Their initial dimensions were 1.524 millimeters in length and 0.254 millimeters in diameter. After 15 minutes in a 0.9% sodium chloride solution, their dimensions changed to 1.27 millimeters in length and 1.016 millimeters in diameter. After 15 minutes in a 0.9% sodium chloride solution, their dimensions changed to 1.27 millimeters in length and 1.016 millimeters in diameter. Borate is an effective antimicrobial. In use, the borate provides resistance to microbial attack of the polysaccharide or other material used for the device.

Removal of Hydrogel Occlusive Devices by Changes in Tonicity

Swelling of hydrogels is often sensitive to changes in pH, temperature and/or tonicity. Shrinkage of gels will occur if it is subjected to an environment outside their optimal swelling conditions. This phenomenon can be used to easily flush an implanted hydrogel from its location. Or a hydrogel implant may be removed using other means after it has been forced to change its dimensions and thereby become less firmly set in place. For example, the implant may be removed by forceps, or surgically.

Changes in pH and temperature should be avoided when flushing a hydrogel implanted in the body, simply because of possible tissue damage. This is especially important in sensitive areas such as the eye or middle ear. Therefore, the safest method for changing dimensions of a hydrogel in vivo will be through alteration of tonicity. Those skilled in the art would recognize that any flexible and very hydrated material such as a hydrogel will collapse if exposed to steep osmotic gradients such as those imposed by hypertonic salt solutions. Very concentrated solutions of salts (for example, sodium chloride) could unfortunately irritate or damage tissues. It has been found that water soluble polymers can substitute for ionic salts to create very hypertonic solutions capable of altering (shrinking) the dimensions of hydrogel materials while remaining gentle enough to use in the body.

Preferably, the water soluble polymer used to change tonicity will be non-ionic. Polymers in this class include polyvinyl alcohol, polyethylene glycol, polyethylene oxide, etc. These can be readily dissolved at high concentration in physiological saline to create safe solutions for use in the body. Alternatively, some biocompatible polymers such as low molecular weight polyethylene glycols are liquids at room temperature; these can also be employed. Preferred polymers are those which are not only water soluble but also are lubricious in nature. Polyethylene glycol is one such example. Polysaccharide polymers are less preferred because, in general, they form very thick solutions in water, even at low concentrations.

Gellan gum was acidified by washing three times with 5% citric acid in water. Resulting acidified gellan powder was subsequently rinsed with water and alcohol and allowed to dry. Acidified powder (15 grams) of gellan gum was dissolved into 100 milliliters of dimethyl sulfoxide to make a 15% solution which was subjected to a vacuum to remove air bubbles. This solution was extruded under air pressure (45-50 pounds per square inch) into 7.5% sodium chloride and 2.5% sodium bicarbonate in water and allowed to incubate for 30 minutes. It was subsequently washed in 10% sodium chloride and then dehydrated in a graded ethanol series. After stretching and drying, they were cut into small pieces representative of an occlusive device.

Dried and cut gellan extrusions were placed into physiological saline and allowed to swell to maximum size, which was measured using a dissecting microscope at 40×magnification. Their dimensions were 2 mm in length and 1.5 mm in diameter. After incubation for 2.5 minutes with 40% polyethylene glycol (average molecular weight 1,000) in physiological saline, their dimensions were again measured. Length was found to be 1.5 mm and diameter was 1.0 mm. This represents a 25% decrease in length and 33% decrease in diameter.

Gellan gum was acidified by washing three times with 5% citric acid in water. Resulting acidified gellan powder was subsequently rinsed with water and alcohol and allowed to dry. Acidified powder (15 grams) of gellan gum was dissolved into 100 milliliters of dimethyl sulfoxide to make a 15% solution which was subjected to a vacuum to remove air bubbles. This solution was extruded under air pressure (45-50 pounds per square inch) into 7.5% sodium chloride and 2.5% sodium bicarbonate in water and allowed to incubate for 30 minutes. It was subsequently washed in 10% sodium chloride and then dehydrated in a graded ethanol series. After stretching and drying, they were cut into small pieces representative of an occlusive device.

Dried and cut gellan extrusions were placed into physiological saline and allowed to swell to maximum size, which was measured using a dissecting microscope at 40× magnification. Their dimensions were 2 mm in length and 1.5 mm in diameter. The fully swollen plugs of sodium gellan were then subjected to dehydration by pure glycerol. After incubation for 2.5 minutes with glycerol, their dimensions had decreased to 1.75 mm in length and 1.0 mm in diameter. This represents a 12.5% decrease in length and a 33% decrease in diameter.

Chelation-Resistant and Triggerably Dissoluble Ionic Gels with Insolubilized Ions

Chelation-resistant (and triggerably dissoluble) ionic gels may be made using insolubilized ions. Devices exposed to chelating agents during their normal use may thus advantageously be made from chelation-resistant materials. Chelation can have a significant effect on the physical properties of gels that are crosslinked by chelatable ions. In the case of occlusive materials to be used in the eye, removal of ions from gels by exposure to chelating solutions, e.g., contact lens cleaners and eye drops, can undesirably affect size and durability of the plug. An increase in chelation resistance enables the creation of chemically durable implants.

Ionic hydrogels of gellan gum, pectinic acids, alginic acids, and the like, typically can crosslink with metal ions, e.g., calcium, magnesium, zinc, copper, barium, iron, aluminum, chromium, and cerium. Metals include, e.g., alkaline earth metals, transition metals, and heavy metals. Metal ions are, in general, easily removed by chelating agents, e.g., sodium citrate or disodium EDTA, both of which are commonly found in certain medical preparations.

But metals that have been complexed with other chemicals to make a mineral are not as easily chelatable. The introduction of a mineral-forming substance into ionic hydrogels may be used to create implants and materials that resist chelation. A mineral-forming substance may be introduced, e.g, into a spin dope or a coagulating bath used for producing these materials. Mineral-forming substances are those substances capable of forming insoluble ionic compounds with metals. Thus the mineral phase may include the organic phase of one or more anionic polymers crosslinked to an inorganic phase of an insoluble metal salt. Minerals are often a combination of oppositely charged substances. Examples of a metal in the mineral phase are calcium, magnesium, zinc, copper, barium, iron, aluminum, chromium, cerium, alkaline earth metals, transition metals, and heavy metals. The mineral phase may be a reaction product of the metal and, e.g., at least one member of the group consisting of silicates, sulfides, halides, oxides, borates, carbonates, sulfates, phosphates, arsenates, vanadates, tungstates, molybdates, hydroxides, and chromates. The degradable, chelation-resistant material may comprise a polysaccharide. Examples of polysaccharides include gellan, polysaccharides closely related to gellan and polysaccharides related to gellan. A mineral-forming substance that is reacted with an ion to form an insoluble compound is referred to as forming a mineral phase, or as creating insolubilized ions. Gels made according to these methods, especially those made with transition metals, may be used to form, e.g, suitable occlusive implants or for long-term occlusion or blockage of a lumen or void. In certain embodiments, these mineral-forming substances may be used by incorporating them so that swelling of gels is not unduly affected by the mineral phase and the mineral phase is not easily removed by chelating agents.

In one process, for example, for example, gellan gum was acidified by washing three times with 5% citric acid in water. Resulting acidified gellan powder was subsequently rinsed with water and alcohol and allowed to dry. Acidified powder (15 grams) was dissolved into 100 milliliters of dimethyl sulfoxide to make a 15% solution which was placed under vacuum to remove air bubbles. The solution was extruded under air pressure (45-50 pounds per square inch) into a 10% aqueous solution of cuprous (copper (I)) chloride. After incubation for 15-30 minutes, extrusions were thoroughly washed in deionized water, stretched, and left exposed to air. Within 1 hour extrusions took on a turquoise color indicative of oxidation of copper(I) ions to copper(II) ions. After drying was complete, extrusions were placed into physiological saline containing 0.025% disodium EDTA. Extrusions swelled to at least 100% their original size and did not lose color. When placed into 5% sodium citrate, color was gradually lost over a 1 hour period, indicating that high concentrations of chelating agents are capable of binding and removing copper from this system. Low concentrations of chelating agents present in a physiological saline solution are essentially ineffective at copper removal, so that these gels were essentially unchelatable when exposed to concentrations of chelating agents that are conventionally found in a solution intended for contact with a patient.

In another process, for example, gellan gum was acidified by washing three times with 5% citric acid in water. Resulting acidified gellan powder was subsequently rinsed with water and alcohol and allowed to dry. Acidified powder (15 grams) was dissolved into 100 milliliters of dimethyl sulfoxide to make a 15% solution which was placed under vacuum to remove air bubbles. The solution was extruded under air pressure (45-50 pounds per square inch) into a 10% aqueous solution of ferrous (iron(II)) sulfate. After incubation for 15-30 minutes, extrusions were thoroughly washed in deionized water and placed in 100% humidity at 65° C. overnight. Upon completion of the oxidation reaction, extrusions had changed from a straw color to brown-green, indicative of oxidation of iron(II) ions to iron(III) ions. After drying, extrusions were placed into physiological saline containing 0.025% disodium EDTA. Extrusions swelled to at least 100% their original size and did not lose color. When placed into 5% sodium citrate, color was gradually lost over a 1.5-2 hour period, indicating that high concentrations of chelating agents are capable of binding and removing iron from this system. Removal of iron by chelating agents was slower than was the case with copper, which is expected as copper has greater affinity for chelating ions than does iron. Low concentrations of chelating agents present in the physiological saline solution are essentially ineffective at ferric ion removal.

A chelation-resistant material may further include unmineralized free metal ion-binding functional groups, so that non-mineralized metals may be complexed thereto, and for subsequent metal-catalyzed degradation. Such gels may be removed as described below, e.g., a gel is exposed to metallic ions, especially iron or copper ions, reaction that bind to the functional groups on the polymer(s) that bind the metal ion. The metal ions are used as catalysts to catalyze oxidation by a peroxide, e.g., benzoyl peroxide or hydrogen peroxide, or ascorbate (vitamin C). Polymers which effectively bind metals usually have amino, carboxyl, phosphate or sulfate functional groups. Covalent crosslinking of such polymers to form hydrogels may therefore be accomplished so as to leave these groups free, or partially free, to interact with metal ions. If polysaccharides are to be used to create gels, therefore, their hydroxyl groups may be utilized in crosslinking reactions instead of other groups such as carboxyls.

Chelation-resistant and triggerably dissoluble ionic gel material may include an acidic polysaccharide treated with acid-catalyzed depolymerization to lower the molecular weight of the acidic polysaccharide. The material may be anisotropically swellable, and may comprise polymers processed into an arrangement of polymers that are substantially parallel to each other. The device may be essentially completely degradable in less than about 5 days to about five years in vitro in a physiological saline solution kept at 37° C.; persons of ordinary skill in these arts will appreciate that all ranges and values between these explicit limits are contemplated, e.g., less than 7 days, 7 days, and two years. One method of using a degradable, chelation-resistant material is to facilitate its removable by exposure to salt-free water, or substantially deionized water. Another method of using a degradable, chelation-resistant material is to take advantage of copper or iron ions contained therein to facilitate gel depolymerization by oxidizing agents such as hydrogen peroxide or ascorbate.

Certain embodiments may be prepared by: (1) selecting a first group of at least one polymer capable of binding metals via free anionic groups (sulfate, carboxylate, phosphate, etc.) or by formation of coordination compounds (for example, iron-chitosan) (2) selecting a second group of at least one polymer sensitive to free radical degradation (3) optionally but preferably selecting the polymers of the first group to have functional groups capable of crosslinking to the first polymer but not to itself; and (4) optionally but preferably designing the gel to have high water content to facilitate fluid flow to deliver transition metal ions and oxidizing agents to the gel interior. A safe and effective means of creating radicals to degrade the resultant gel is via oxidation using ascorbate or peroxide with, e.g., ferric or cupric ions as catalysts.

Some embodiments are gels made by crosslinking a first polymer with a second polymer that is triggerably degradable by metal-catalyzed oxidation. The crosslinking of the first and second polymer creates a hydrogel but the degradation of the second polymer causes the gel to degrade. Either the first or the second polymer has functional groups that are capable of binding a metal ion. The crosslinking may be performed by, e.g., an acid-catalyzed esterification of hydroxyl and carboxyl groups. To make the gel, the first and the second polymer may be mixed together and exposed to heat under acidic conditions to crosslink their functional groups to each other or to a crosslinking agent. An embodiment of such a material is: a first polymer capable of binding metals via free anionic groups (sulfate, carboxylate, phosphate, etc.) or by formation of coordination compounds (for example, iron-chitosan); a second polymer that is sensitive to free radical degradation. The polymers not sensitive to free radical degradation preferably have only one type of functional group capable of crosslinking (i.e. it cannot be crosslinked to itself). The resultant hydrogels may have a high water content to facilitate fluid flow necessary to deliver transition metal ions and oxidizing agents to the gel interior.

Occlusive devices could be made with a chelation-resistant material by using the material in a mold or other process that is used to make conventional devices based on collagen or other materials. Certain embodiments include a device for occluding a duct, passage, wound or orifice, the device including an introducible portion that is introducible into the duct, passage, wound or orifice to at least partially block movement of a fluid, wherein at least a part of the introducible portion comprises a degradable, chelation-resistant material that is essentially completely degradable in less than about 365 days, about 180 days, about 90 days, about 7 days, or between about 1 day and about five years in vitro in a physiological saline solution kept at 37° C. Alternatively, the device can be formed to essentially last the lifetime of the patient. Persons of ordinary skill in these arts will appreciate that all ranges and values within the explicitly articulated range are contemplated.

Some embodiments of crosslinked chelation-resistant gels for free radical-triggered dissolution involve crosscarmellose sodium and carboxylic acid-crosslinked water soluble polymers. In principle any polysaccharide capable of forming a hydrogel can be treated according to the following methods, as can many synthetic polymers such as polyvinyl alcohol. The only requirements are presence of functional groups for crosslinking reactions and ability to be degraded by free radical mechanisms.

Another embodiment is a material for occluding a lumen or void, e.g., duct, passage, orifice, or void created by a wound, the device comprising an introducible portion that is introducible into the lumen or void to at least partially block movement of a fluid, wherein at least a part of the introducible portion comprises a polysaccharide and a mineral phase that comprises a metal.

Controllably Degradable Materials and Devices

Some embodiments are implantable devices and materials that are made of short-term degradable materials. Depolymerized gellan and related polysaccharides such as welan, S-88, S-198 or rhamsan gums are examples of such materials. Gellan may be depolymerized to achieve a desired rate of degradation. For example, to achieve a rapid dissolution time of 5-10 days, the molecular weight of gellan gum may be lowered.

Referring to FIG. 1, it is evident that the molecular weight of gellan can be very high. One method for lowering the molecular weight is with acid-catalyzed depolymerization. Most polysaccharides, when exposed to strong acids, will undergo hydrolysis of glycosidic bonds. This process is accelerated by heat, oxygen and/or water. Protonated uronic acid residues can also participate by catalyzing depolymerization through intramolecular catalysis. For these reasons, neutral polysaccharides typically degrade more slowly at low pH than do acid polysaccharides. Degradation of free acid forms of polysaccharides is referred to herein as autocatalytic hydrolysis. Dissolution times may thus be adjusted by controlling the amount of depolymerization, which may be performed by controlling the depolymerization conditions, e.g., heat, oxygen, and/or water. The Swellable Temporary Punctum Plug example, below, describes experiments that document how degradation can be controlled using these techniques.

Among acid polysaccharides, self-catalyzed degradation is related to the relative abundance of uronic acid residues in the polymer chain. Glycosidic linkages between uronic acid residues are more resistant to hydrolysis than are those between neutral residues. Polysaccharides composed of only uronic acid residues will thus degrade more slowly at low pH than will polysaccharides with neutral and acidic residues. Gellan possesses one uronic acid residue to every three neutral residues. It is therefore quite sensitive to autocatalytic hydrolysis. In principle, all acidic polysaccharides and their semisynthetic derivatives can be depolymerized by acidification and heat treatment with water and/or oxygen. Depolymerization would be influenced by the nature of glycosidic bonds among saccharide residues as well as the amount of uronic acid residues present in the polymer.

Autocatalytic hydrolysis can be performed at various steps in the process of preparing a material or a device. For example, gellan may be treated while in solution before forming the gellan into a material or device. Alternatively, the treatments may be performed on gellan powders, fibers, filaments and films. A requirement is that water or oxygen should be capable of reacting with the polymer, preferably in a uniform manner so as to ensure a consistent product. Low reaction temperatures are preferred as they allow easy control over the extent of degradation. Reactions normally take 6-48 hours to complete.

Acidified gellan, regardless of its extent of depolymerization, can be dissolved in polar organic solvents to fabricate extrusions. Coagulation baths typically consist of aqueous solutions of organic acids, inorganic acids or basic salts of monovalent cations. After thorough washing to remove excess acid or salt, extrusions are easily stretched to over twice their original length, facilitated by the lack of ionic crosslinking of gellan polymer chains. Hydrogen bonding among chains of gellan polymer are, in this case, largely responsible for gel formation. Should depolymerization of stretched, extruded material be desired, acidified (not neutralized) gellan extrusions are incubated at ≧65° C. in the presence of air and/or water vapor. After 6-48 hours, the acidic extrusions are neutralized in a solution containing alkali salts. It is preferable to use either excess salt or 50-70% alcohol in the alkali bath to suppress swelling which would ruin stretching-induced molecular orientation. They gel weakly upon contact with saline. Using this method, it is possible to make strong extrusions which are easily oriented by stretching but which will result in only weak gels once inserted in the body.

Depolymerized gellan may be made that is stable in saline for 1 hour to only slightly less than that which is possible without depolymerization treatment. Similar polymers such as alginate have duration times in vivo for over 5 years, so gellan could be made with a similar durability. Durability depends on extent of polymer protonation and duration/temperature at which autocatalytic degradation proceeded. In saline, depolymerized material tends to fragment into increasingly smaller pieces. This indicates that molecular weight has been reduced via hydrolysis. In contrast, sodium gellan which has not been subjected to depolymerization is stable in saline for an indefinite time so long as it is not subjected to microbial attack.

For example, to show the creation of rapidly degradable depolymerized polymers made from polysaccharides, gellan gum was acidified by washing three times with 5% citric acid in water. The resulting acidified gellan powder was subsequently rinsed with water and alcohol and allowed to dry. Acidified powder (15 grams) of gellan gum was dissolved into 100 milliliters of dimethyl sulfoxide to make a 15% solution which was subjected to a vacuum to remove air bubbles. This solution was extruded under air pressure (45-50 pounds per square inch) into a coagulation bath consisting of 10% citric acid in distilled water.

Extrusions were removed from the coagulation bath, washed three times in distilled water and dehydrated through a graded alcohol to series up to 91% alcohol. Once removed from 91% alcohol, extrusions were placed on a ruler, measured, and then stretched to twice their original length and allowed to dry. Once dried, extrusions were placed in an incubation chamber at 65° C. and 100% humidity for 0, 6, 8, 18 and 48 hours. Experimental groups consisted of extrusions treated at 65° C. and 100% humidity for the four time intervals; untreated extrusions acted as controls. Samples from each group were air-dried after incubation to remove excess water and then dissolved in DMSO to make a 2.5% solution. Gellan, free acid (2.5%) in DMSO from each sample group was tested for viscosity using a falling ball viscometer at 22° C. Results were as follows:

| Depolymerization Time (hours) | Solution Viscosity (centipoises) |
|---|---|
| 0 hr | 244.25 cP |
| 6 hr | 61.91 cP |
| 8 hr | 59.69 cP |
| 18 hr | 32.43 cP |
| 48 hr | 24.31 cP |

As another example to demonstrate the use of depolymerized gellan gum as a temporary occlusive device, gellan gum was acidified by washing three times with 5% citric acid in water. The resulting acidified gellan powder was subsequently rinsed with water and alcohol and allowed to dry. Acidified powder (15 grams) of gellan gum was dissolved into 100 milliliters of dimethyl sulfoxide to make a 15% solution which was subjected to a vacuum to remove air bubbles. This solution was extruded under air pressure (45-50 pounds per square inch) into a coagulation bath consisting of 10% citric acid in distilled water. Extrusions were removed from the coagulation bath, washed three times in distilled water and dehydrated through a graded alcohol to series up to 91% alcohol. Once removed from 91% alcohol, extrusions were placed on a ruler, measured, and then stretched to twice their original length and allowed to dry. Once dried, extrusions were placed in an incubation chamber at 65° C. and 100% humidity for 6.75 hours. Extrusions were then neutralized with a hypertonic aqueous solution of sodium bicarbonate and sodium chloride, rinsed in hypertonic sodium chloride and dehydrated through a graded ethanol series. Plugs were fabricated by cutting extrusions in sections approximately 1.5 millimeters long.

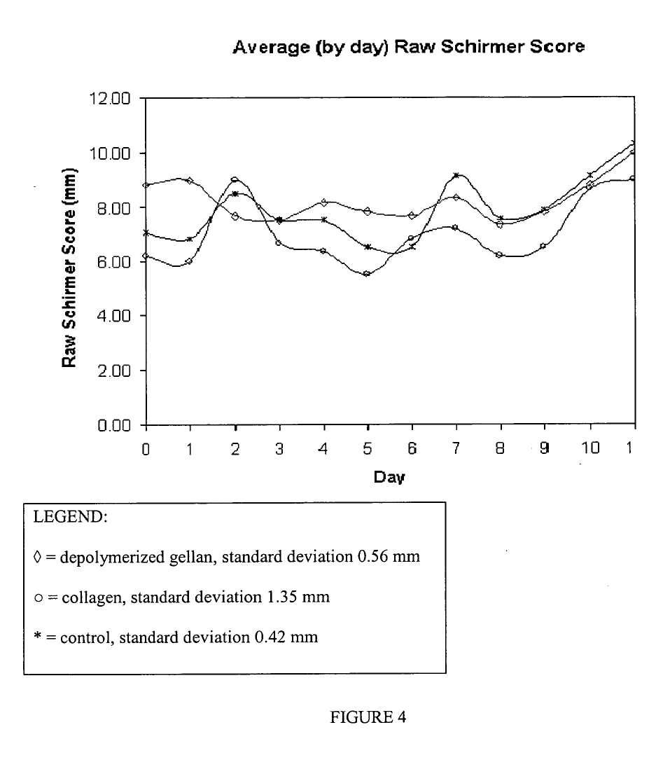

Plugs were sterilized with ethylene oxide and implanted into the nasolacrimal system of rabbits. The protocol used 12 rabbits, with the right eyes of these rabbits occluded with a temporary punctum plug, and the left eye was left unoccluded. Six days of baseline data was gathered for each rabbit, in both eyes, prior to occlusion. Six rabbits received Collagen plugs in the right eye, and the remaining six rabbits received depolymerized gellan plugs in the right eye. All left eyes were left unoccluded for the duration of the study. Each day tear film was assessed using Schirmer strip scores for both eyes, in all rabbits, and recorded as the length in millimeters of wetted strip material. The animals were also observed for any signs of irritation, epiphora, erythema, pruritus, infection, or swelling, which would indicate removal of the insert. There were no observed cases of any of these conditions in any of the animals. After the data was collected, it was analyzed in the following manner, see FIG. 4, The average daily raw Schirmer score was calculated for three different data sets, the collagen occluded eyes (six points per day), the depolymerized gellan occluded eyes (six points per day), and the unoccluded control eyes (twelve points per day). The daily standard deviation was also calculated, and averaged across all days. The daily averages were then plotted on a graph to compare the two occlusive methods with the unoccluded control group of eyes.

As is evident from the data of FIG. 4, depolymerized gellan gum can serve as a temporary plug to block the flow of fluid through an opening or duct. It performed more consistently than did the currently accepted practice of using collagen as an occlusive material.

Triggerable Dissolution of Occlusive Medical Device Implants

Metal-catalyzed oxidation may be used to triggerably dissolve a polymeric material. Free metal ions are associated with the polymer before, during, or after the formation of the gel. The metal ions are used as catalysts to catalyze oxidation by a peroxide, e.g., benzoyl peroxide or hydrogen peroxide, or ascorbate (vitamin C). Polymers which effectively bind metals usually have amino, carboxyl, phosphate or sulfate functional groups. Covalent or other crosslinking of such polymers to form hydrogels may therefore be accomplished so as to leave at least some functional groups free to bind metal ions. If polysaccharides are to be used to create gels, therefore, their hydroxyl groups may be utilized in crosslinking reactions instead of other groups such as carboxyls. Some or all of the polymers or materials in a gel or hydrogel may be used to capture the free metal ions. As set forth in greater detail in U.S. Patent Application Ser. No. 60/557,368, covalently crosslinked chelation-resistant gels for triggerable dissolution may be made by crosslinking a first polymer with a second polymer that is triggerably degradable by metal-catalyzed oxidation. Such materials may be made into a device for occluding a duct, passage, wound or orifice as described herein, or as referenced herein.

In some embodiments, the crosslinking of a first and a second polymer may create a hydrogel, while degradation of the second polymer causes the gel to degrade. Either the first or the second polymer has functional groups that are capable of binding a metal ion. The crosslinking may be performed by, e.g., an acid-catalyzed esterification of hydroxyl and carboxyl groups. To make the gel, the first and the second polymer may be mixed together and exposed to heat under acidic conditions to crosslink their functional groups to each other or to a crosslinking agent.

Chemical removal may be effected by oxidation using peroxides (e.g., benzoyl peroxide or hydrogen peroxide) or ascorbate (vitamin C). Transition metals, especially iron and copper ions, may be used as catalysts for the reaction. In topical applications, a ferrous chloride-3% hydrogen peroxide system can be used for very rapid degradation of susceptible hydrogels. However, hydrogen peroxide typically cannot be used in the eye; therefore ferric chloride/cupric chloride-ascorbate system is advantageous. Removal of subpunctal devices may be achieved in the following manner: (1) Flushing of the gel with an isotonic or slightly hypertonic solution containing transition metal ions, ferric and cupric ions being preferred. The anionic groups will bind metal ions, atomically dispersed throughout the gel; (2) Rinsing of the surrounding tissues with neutral buffered saline or water for injection, not allowing gels to be exposed to chelating agents such as disodium EDTA or sodium citrate; and (3) Application of diluted ascorbic acid or ascorbic acid salts to the gel. Periodic application will oxidize the gel, rendering it brittle and mechanically weak enough to crumble apart. Devices made from gels crosslinked with iron or copper ions or with insolubilized iron or copper ions are advantageous for this removal method as the addition of further salt solutions is not necessary. Flushing with oxidizing agents such as ascorbate or hydrogen peroxide would be sufficient to oxidize and degrade the device.

An embodiment is a device for occluding a lumen or void, e.g., a duct, passage, orifice, or void created by a wound, the device comprising an introducible portion that is introducible into the duct, passage, wound or orifice to at least partially block movement of a fluid through the passage, wherein at least a part of the introducible portion comprises at least a first polymer that is triggerably degradable by metal-catalyzed oxidation. In certain embodiments, at least a part of the introducible portion further comprises a second polymer, wherein at least one of the first and the second polymer comprises at least one functional group capable of binding a metal ion. In some cases, the first and the second polymer are crosslinked by acid-catalyzed esterification of hydroxyl and carboxyl groups. The polymers may comprise a polysaccharide, e.g., gellan, welan, S-88, S-198, a rhamsan gum. The polymers may comprise, e.g., at least one member of the group consisting of alginate, curdlan, carboxymethylcellulose, crosscarmellose, poly(acrylic acid), xanthan, carrageenan, carboxymethyl chitosan, hydroxypropyl carboxymethyl cellulose, pectin, gum Arabic, karaya gum, psyllium seed gum, carboxymethyl guar, and mesquite gum. The material may include an acidic polysaccharide treated with acid-catalyzed depolymerization to lower the molecular weight of the acidic polysaccharide. The material may comprise a metallic ion. The material may be anisotropically swellable, and may comprise polymers processed into an arrangement of polymers that are substantially parallel to each other.

As set forth in detail, herein, and in U.S. Patent Application Ser. No. 60/557,368, devices may be removed using metal-catalyzed oxidation. One method of removing a device for occluding a duct, passage, wound or orifice comprises exposing the device to metal-catalyzed oxidation to degrade a material in the device to facilitate removal of the device from the duct, passage, wound or orifice. Such a device may have metal ion-binding functional groups to facilitate such catalytic oxidation. The device may comprise an introducible portion that is introducible into the duct, passage, wound or orifice to at least partially block movement of a fluid, wherein at least a part of the introducible portion comprises the material.

In one embodiment, an occlusive device is removable by a metal-catalyzed oxidative processes, e.g., by exposure to a peroxide to effectively dissolve or disintegrate the device or to make the device brittle and readily subject to break-up by mechanical forces. For example, gellan gum was acidified by washing three times with 5% citric acid in water. Resulting acidified gellan powder was subsequently rinsed with water and alcohol and allowed to dry. Acidified powder (15 grams) of gellan gum was dissolved into 100 milliliters of dimethyl sulfoxide to make a 15% solution which was subjected to a vacuum to remove air bubbles. This solution was extruded under air pressure (45-50 pounds per square inch) into 10% ferrous sulfate in water and allowed to incubate for 30 minutes. It was subsequently washed three times in distilled water to remove any free ions. After washing extrusions were placed in an aqueous solution of 3% hydrogen peroxide. Within 1 minute the gel extrusions became very brittle and could not be manipulated with forceps without fracturing.

And, for example, gellan gum was acidified by washing three times with 5% citric acid in water. Resulting acidified gellan powder was subsequently rinsed with water and alcohol and allowed to dry. Acidified powder (15 grams) was dissolved into 100 milliliters of dimethyl sulfoxide to make a 15% solution which was placed under vacuum to remove air bubbles. The solution was extruded under air pressure (45-50 pounds per square inch) into a 10% aqueous solution of cuprous (copper (I)) chloride. After incubation for 15-30 minutes, extrusions were thoroughly washed in deionized water, stretched, and left exposed to air. Within 1 hour extrusions took on a turquoise color indicative of oxidation of copper(I) ions to copper(II) ions. Extrusions were transferred to an aqueous solution of 3% hydrogen peroxide and allowed to incubate for 1-5 minutes. When removed from the hydrogen peroxide solution, the extrusions were easily fractured as they had become embrittled. Microscopic examination at 40× revealed that the surface had become pitted with chevron-shaped crevices which were especially noticeable if attempts were made at stretching.

And, for example, sodium carboxymethylcellulose was acidified by washing three times with 5% citric acid in 70% isopropyl alcohol. The resulting acidified carboxymethylcellulose powder was subsequently rinsed with 70% isopropyl alcohol and allowed to dry. Acidified powder (15 grams) of carboxymethylcellulose was dissolved into 100 milliliters of dimethyl sulfoxide to make a 15% solution and placed under vacuum to remove air bubbles. This solution was extruded under air pressure (45-50 pounds per square inch) into 70% isopropyl alcohol acidified with 10% citric acid. After washing in progressively concentrated alcohol solutions, extrusions were stretched, dried and placed under nitrogen atmosphere and cured for 24 hours at 65° C. After curing, extrusions were placed into a 10% solution of ferric (iron(III)) chloride and allowed to incubate for 30 minutes. They were subsequently washed three times in distilled water to remove any free ions. Extrusions were then placed into dilute aqueous ascorbic acid (ca. 1-2%) and incubated for 30 minutes. After this time extrusions were stronger than were oxidized gellan extrusions but became brittle enough that fracture would occur on bending or stretching

Fluidic Occlusive Elements and Materials

Occlusive elements may be made by introducing a fluidic material a to a space to be occluded, and allowing the material to hydrate to a more viscous condition. Production of fluid or otherwise flowable gels is straightforward. Polymers which are soluble in hot water but which gel when cooled are used. Briefly, a polymer such as gellan gum, a polysaccharide closely related to gellan, or a polysaccharide related to gellan gum is dispersed in cold water and heated until a weak solution is made. As the solution is cooling, it is beaten, stirred or otherwise vigorously agitated such that when room temperature is reached, a fluid remains. These fluids are typically non-viscous and show non-Newtonian flow. Fluid gels are then concentrated by evaporation, filtration or centrifugation until a solids content of at least about 10% is achieved. The suspension can then be extruded into a coagulating bath to form filaments. The degradation rate of these fluids may be controlled by adjusting the concentration of the polymer and the degree of mechanical agitation of the polymers.

When filaments are dried and placed in the body, they hydrate rapidly, forming a viscous fluid which resists flow. Various compositions have been made according to these methods that degrade in between 4 hours and 72 hours when implanted into a nasolacrimal duct of a human patient. Persons of ordinary skill in these arts, after reading this disclosure, will be able to prepare such implantable compositions with a predetermined degradation time.

An embodiment is a medical device for occluding a void or lumen, the device comprising an aggregation of small particles introducible into the void or lumen to form a viscous suspension to at least partially block movement of a fluid through the passage. The small particles may comprise a polysaccharide. The small particles may comprise a polymer such as gellan gum, a polysaccharide closely related to gellan, or a polysaccharide related to gellan gum. The device may be essentially completely degradable in vitro in a physiological saline solution maintained at 37° C. in less than about 7, 5, 3, or 0.5 days. The aggregation may be, e.g., a filament. The device, or a portion thereof, may further comprise a therapeutic agent with/without DMSO and/or MSM. Other examples of using an occlusive device are provided herein.

Materials of Water-Soluble Polymers Which Gel Under Physiological Conditions

Polysaccharides of the gellan family (gellan, welan, S-88, S-198 or rhamsan gums) can be fabricated into solid materials which imbibe water and gel in the presence of physiological fluid. In deionized water or in aqueous solutions of chaotropic agents such as tetramethylammonium chloride, no gelation occurs—the polymers remain soluble. Gelation in physiological fluids is believed to be due to the presence of sodium ions, which can act as kosmotropic agents, i.e., agents which have strong interactions with water molecules and act to maintain gel structure. Under physiological conditions, devices made with sodium gellan swell up to 3 times their original size and effectively fill spaces into which they are placed.

If left in physiological saline, sodium gellan gels will not degrade even over extended periods. Gels are nevertheless quite soluble when contacted with deionized water. Solubility can be decreased to some extent by addition of polymers which can form hydrogen bonds with sodium gellan (99% hydrolyzed polyvinyl alcohol and tamarind seed gum are primary examples). This is analogous to the calcium-alginate-PVA gel system used to sequester metals or encapsulate microorganisms (Klimiuk and Kuczajowska-Zadrozna, 2002; Pattanapipitpaisal, Brown and Macaskie, 2001; Micolay et al., 2003). A factor influencing water solubility is the freedom of the gel to expand. For example, a sodium gellan gel placed unconstrained in water at room temperature will start to dissolve after 5-10 minutes. If the gel is constrained in tubing such that its lateral dimensions are fixed, it will not dissolve in ion-free water even after 24 hours. Without being committed to a particular theory of operation, it is believed that constraint results in a gel concentration that is greater than is its solubility in water.

Furthermore, it has been found that if water is injected into or around a constrained gel and is allowed to flow swiftly, sodium gellan gels will shrink in dimensions. For constrained sodium gellan gels solubility in water appears to be a function of velocity of water moving through and around the gel. Moving water is able to carry soluble polymer molecules away from the main body of the gel much more effectively than is still or slowly moving water. These results show that implants made of sodium gellan can be stable unless intentionally removed with water through irrigation. Additional details are set forth in U.S. Patent Application Ser. No. 60/557,368.

An embodiment is a device for occluding a lumen or void, the device including an introducible portion that is introducible into the lumen or void to at least partially block movement of a fluid therethorugh, wherein at least a part of the introducible portion comprises at least one polysaccharide in the group consisting of gellan, welan, S-88, S-198 and rhamsan gum. The polysaccharide may include, e.g., an acidic polysaccharide treated with acid-catalyzed depolymerization to lower the molecular weight of the acidic polysaccharide. The polysaccharide may also include a metallic ion. The polysaccharide may also include an arrangement of polymers that are substantially parallel to each other.

Gellan gum was acidified by washing three times with 5% citric acid in water. Resulting acidified gellan powder was subsequently rinsed with water and alcohol and allowed to dry. Acidified powder (15 grams) of gellan gum was dissolved into 100 milliliters of dimethyl sulfoxide to make a 15% solution which was subjected to a vacuum to remove air bubbles. This solution was extruded under air pressure (45-50 pounds per square inch) into 10% citric acid in water and allowed to incubate for 30 minutes. It was subsequently washed three times in distilled water to remove any free ions. Extrusions were dehydrated in a graded alcohol series to 91% alcohol and subsequently stretched to twice their original length. They were allowed to air dry. After drying extrusions were placed into a saturated sodium carbonate solution for 20 minutes followed by a saturated sodium chloride solution for another 20 minutes. After rinsing twice in 70% alcohol for 20 minutes each and 91% alcohol for 20 minutes, extrusions were allowed to air dry.

Extrusions were placed into distilled water to assess neutralization, as sodium gellan, but not acidic gellan, is very soluble in distilled water. After 10 minutes extrusions were dissolved, indicating neutralization had been achieved. At no time during neutralization did extrusions become soft or swell, indicating that orientation had been maintained. Prototype occlusive devices were fabricated by cutting neutralized extrusions into cylindrical pieces. Their dry dimensions were 1.524 millimeters in length and 0.254 millimeters in diameter. Once placed into physiological saline and allowed to swell to their maximum extent, they had dimensions of 1.27 millimeters in length and 1.016 millimeters in diameter.

Methods of Making Hydrophilic Extrusions, Fibers and Monofilaments Incorporating Carboxymethylcellulose

A biocompatible and effective crosslinked gelation system involves making gels of sodium carboxymethylcellulose-croscarmellose sodium. Croscarmellose sodium is a cross linked polymer of carboxymethyl cellulose sodium. Crosslinking makes it an insoluble, hydrophilic, highly absorbent material, resulting in excellent swelling properties, and its fibrous nature gives it water wicking capabilities. Croscarmellose sodium is useful for drug dissolution and has rapid disintegration characteristics, thus improving bioavailability of formulations. Such gels are useful for forming medical devices for occluding lumens and voids in a patient.

As set forth in detail in U.S. Patent Application Ser. No. 60/557,368, materials and devices may be made using hydrophilic extrusions, fibers, and monofilaments incorporating carboxymethylcellulose. One such embodiment is a method of making an implant comprising a degradable portion that comprises crosscarmellose prepared by acidification of a free acid of carboxymethylcellulose. Acidification displaces neutralizing ions (K⁺ or Na⁺), thereby causing carboxymethylcellulose to behave as an anionic polysaccharide such that it can be dissolved into polar organic solvents such as DMSO or N,N-dimethylacetamide. Dissolution in DMSO, for instance, allows for much higher concentrations than is possible in water, especially if the solution is heated. The concentrated solution can then be used to fabricate extrusions in the form of fibers or monofilaments whose mechanical properties far exceed those of fibers spun from aqueous solutions. It can reasonably be expected that any acidic polysaccharide (having COOH functional groups) could be treated this way. Once the material has been shaped to its final form, e.g., by extrusion, it can be internally crosslinked by methods already known to the arts. U.S. Pat. No. 3,379,720 discloses a method for modifying water-soluble polymers such as carboxymethylcellulose to render them insoluble in water. In this Patent Letters is disclosed a method of forming a device such as a fiber or monofilament which can then be cured to make it insoluble in water as described in U.S. Pat. No. 3,379,720.

If carboxymethylcellulose (FIG. 2) is acidified and heated, a fraction of carboxymethyl groups, which are acidic functional groups, can esterify to —OH functional groups which are present in many water-soluble polymers. Remaining acidic groups can be readily neutralized with alkali. The formation of the —OH functional groups is useful for preparing them for reaction with other functional groups, e.g., —COOH groups in an acid-catalyzed dehydration step.

Occlusive or blocking implants and devices can be made, starting with extrusions of carboxymethylcellulose, followed by treatments to form croscarmellose. Sodium carboxymethylcellulose extruded from water forms fragile and weak gels, these being difficult to handle. It has been found the acidification of carboxymethylcellulose to its free acid allows it to become soluble in polar organic solvents such as DMSO or N,N-dimethylacetamide. Extrusions made from carboxymethylcellulose-DMSO solutions possess reasonable strength. If cured for 12-48 hours at 65° C. in nitrogen, extrusions become very strong. Acidification and associated crosslinking likely raised molecular weight of the carboxymethylcellulose in a manner similar to curing of a thermoset polymer to form a material that can be characterized as a crosslinked croscarmellose. Neutralizing unreacted acidic groups with alkali results in favorable swelling properties. Extrusions are stable in the presence of chelating agents and at any pH likely to be encountered in the body. Croscarmellose sodium can bind ferrous, ferric and cupric ions and is sensitive to oxidative degradation.

Alternatively, polysaccharide films, fibers or filaments can be carboxymethylated by reaction with monochloroacetic acid or its alkali metal salts and then heated to effect crosslinking. For example, acidic gellan gum can be dissolved in DMSO and extruded into an aqueous solution of monochloroacetic acid. The extrusion gels in the presence of acids and by reaction with monochloroacetic acid functional groups capable of forming crosslinks are introduced. By heating in the presence of an inert gas such as nitrogen or argon, crosslinks are formed in the same manner as when synthesizing croscarmellose sodium. Unreacted carboxyl groups can then be neutralized in alcoholic solutions of alkali metal hydroxides or in saturated aqueous solutions of alkali metal carbonates or bicarbonates.

Examples of gels containing crosslinked croscarmellose include, but are not limited to, the following examples. For example, Gellan gum was acidified by washing three times with 5% citric acid in water. Resulting acidified gellan powder was subsequently rinsed with water and alcohol and allowed to dry. Sodium Carboxymethylcellulose was also acidified by washing three times with 5% citric acid in 70% isopropyl alcohol. The resulting acidified carboxymethylcellulose powder was subsequently rinsed with 70% isopropyl alcohol and allowed to dry. Acidified powder (15 grams) of gellan gum was dissolved into 100 milliliters of dimethyl sulfoxide to make a 15% solution. Likewise, acidified powder (15 grams) of carboxymethylcellulose was dissolved into 100 milliliters of dimethyl sulfoxide to make a 15% solution. The two solutions were mixed to a ratio of 5:1 acidified gellan:acidified carboxymethylcellulose and was placed under vacuum to remove air bubbles. The solution was extruded under air pressure (45-50 pounds per square inch) into a 10% citric acid coagulation bath. Extruded material was collected and washed three times for 10 minutes in 70% isopropyl alcohol. Drying was performed at room temperature. Upon drying, extruded material was cured under nitrogen at 65° C. for 24 hours. At that time the material was removed and incubated in distilled water alone or distilled water after washing with aqueous solutions of 2.5% sodium citrate or 2.5% sodium bicarbonate. The material swelled but did not dissolve in these media. Fibrillation or formation of small fibrils on the surface of extrusions upon manipulation was quite marked.

As another example, Gellan gum was acidified by washing three times with 5% citric acid in water. Resulting acidified gellan powder was subsequently rinsed with water and alcohol and allowed to dry. Sodium Carboxymethylcellulose was also acidified by washing three times with 5% citric acid in 70% isopropyl alcohol. The resulting acidified carboxymethylcellulose powder was subsequently rinsed with 70% isopropyl alcohol and allowed to dry. Acidified powder (15 grams) of gellan gum was dissolved into 100 milliliters of dimethyl sulfoxide to make a 15% solution. Likewise, acidified powder (15 grams) of carboxymethylcellulose was dissolved into 100 milliliters of dimethyl sulfoxide to make a 15% solution. The two solutions were mixed to a ratio of 5:1 acidified gellan:acidified carboxymethylcellulose and was placed under vacuum to remove air bubbles. To this solution was added 1 gram of ferric chloride, which dissolved readily in DMSO, turning the solution bright yellow. The solution was extruded under air pressure (45-50 pounds per square inch) into a 1% aqueous ferric chloride coagulation bath. Extruded material was collected and washed three times in 5% citric acid for 30 minutes each wash. A graded series of increasingly concentrated isopropyl alcohol was used to remove any remaining citric acid and water. Drying was performed at room temperature. Upon drying, extruded material was cured under nitrogen at 65° C. for 24 hours. At that time the material was removed and incubated in distilled water alone or distilled water after washing with aqueous solutions of 2.5% sodium citrate or 2.5% sodium bicarbonate. The material swelled but did not dissolve in these media. Upon swelling no fibrillation could be observed and the material was completely insoluble in water.

As another example, a solution containing 5% agarose and 2.5% sodium carboxymethylcellulose in hot water was used to cast a film, which gelled upon cooling. The film was soaked in 10% citric acid in 70% isopropyl alcohol for 1 hour. After being washed three times in 70% alcohol, the film was dried and cured for 24 hours at 65° C. under nitrogen. The film was then removed, neutralized in 2.5% sodium bicarbonate, and placed in water and allowed to swell. It did not dissolve when the water temperature was raised to 100° C.

As an additional example, sodium carboxymethylcellulose was acidified by washing three times with 5% citric acid in 70% isopropyl alcohol. The resulting acidified carboxymethylcellulose powder was subsequently rinsed with 70% isopropyl alcohol and allowed to dry. Acidified powder (15 grams) of carboxymethylcellulose was dissolved into 100 milliliters of dimethyl sulfoxide to make a 15% solution and placed under vacuum to remove air bubbles. This solution was extruded under air pressure (45-50 pounds per square inch) into 70% isopropyl alcohol acidified with 10% citric acid. After washing in progressively concentrated alcohol solutions, extrusions were stretched, dried and placed under nitrogen atmosphere and cured for 24 hours at 650° C. After curing, 2.5% sodium bicarbonate was used to neutralize any remaining acid groups. Extrusions were very strong and swelled 50-100% in physiological saline.

Materials Made by Esterification of Carboxylic Acids

Hydrogels can also be made by crosslinking of hydroxyl functional groups on water-soluble polymers with crosslinking molecules having carboxylic acid functional groups, e.g, citric acid or butanetetracarboxylic acid (BTCA). Effectiveness of crosslinking molecules depends upon their ability to form no fewer than two cyclic anhydrides. Likewise, crosslinking polymers can be used in place of crosslinking molecules, providing they have carboxylic acid groups capable of forming no fewer than two cyclic anhydrides e.g., polymaleic acid or polymaleic anhydride. Alternatively, the water-soluble polymers may have carboxyl groups that are reacted with hydroxyl-bearing crosslinkers. Or the polymers may have both hydroxyl and carboxyl groups. Catalysts such as sodium hypophosphite or sodium salts of fumaric, maleic or itaconic acid may be employed. Such materials are useful as medical implants. This system is safer than alternative crosslinking systems, e.g., using gluteraldehyde, epichlorohydrin, etc. Other crosslinking schemes may be used, e.g., as described by Greg T. Hermanson in Bioconjugate techniques, Academic Press (1996, ISBN: 012342335X).

Crosslinking may be effected at elevated temperatures (50° C.) through formation of two cyclic anhydrides by the sequential reaction of three carboxylic acid functional groups. For crosslinking to occur there are at least three acid functional groups on a crosslinking molecule—two of which will form crosslinks by esterification with hydroxyl groups of a polymer and one of which will remain free to be neutralized later with alkali. Metal binding capabilities of the water-soluble polymer are therefore not altered with this crosslinking method, they are enhanced.

This property can be used to advantage when gels are to serve as occlusive or blocking materials. For example, alginate plugs could be crosslinked with ester bonds through reaction with cyclic anhydrides. Carboxyl groups of uronic acids present in the alginate polymer would remain unreacted as would no less than one carboxyl group on the crosslinking molecule. These unreacted carboxyl groups could be neutralized through binding of metals known for their antimicrobial activity-namely silver, cerium, copper or zinc. Should removal of plugs be necessary, metal ions present in the gel could be displaced using copper or iron salt solutions to catalyze free radical depolymerization by peroxide or ascorbate.

As an example, gellan gum was acidified by washing three times with 5% citric acid in water. Resulting acidified gellan powder was subsequently rinsed with water and alcohol and allowed to dry. Acidified powder (15 grams) was dissolved into 100 milliliters of dimethyl sulfoxide to make a 15% solution which was placed under vacuum to remove air bubbles. The solution was extruded under air pressure (45-50 pounds per square inch) into a solution of 6.5% citric acid and 6.5% sodium fumarate in hot water. After incubation in the hot solution for 5 minutes, extrusions were removed and allowed to air dry at room temperature.

After drying, extrusions were heated to 180° C. in the absence of oxygen for periods of 3-5 minutes followed by washing in 2.5% sodium bicarbonate to neutralize remaining acid. Extrusions were washed in water, dehydrated through a graded ethanol series, stretched and dried. Once dry, extrusions were placed in either distilled water or physiological saline containing 0.025% disodium EDTA. Swelling in both media resulted in gels approximately 3 times their original diameter with no dissolution after 1 hour.

Devices and Uses

Set forth herein are a variety of materials and methods for forming implantable materials and devices. The materials may, accordingly, be used to form implants and other devices. Implantable means a material suitable for introduction into a patient or onto a patient. An implant may thus be disposed, for example, entirely within a patient or partially inside a patient, e.g., in an opening of a patient, with a portion of the device not penetrating the patient. Examples of implants are a composition for oral or suppository introduction into the patient, a device placed in or applied to a wound, and a material placed into a naturally-occurring opening in a patient, for example an ear canal. The sue of the materials and methods set forth herein are also contemplated for topical application to a patient, e.g., on a patient's skin.

Implants have many uses known to persons of skill in these arts. Uses include occlusion (essentially complete blockage) of an opening, blockage of an opening, and drug delivery. For example, a drug or other therapeutic substance may be associated with the implant, which may serve as a delivery vehicle for delivery or release of the drug. For example, a material may be formed into a swellable plug and introduced into a wound site, where it swells to become firmly set in the wound. Many types of implants are known to persons of ordinary skill in these arts.

Materials and devices set forth herein may be prepared, as appropriate, in a variety of forms, including gels, crosslinked gels, and powders. Other forms include fibers, filaments, and films. Processing steps may include, as appropriate, molding, extrusion, and polymerization. Materials and devices set forth herein may be prepared, as appropriate, in combination with other polymers and materials. For example, fillers, plasticizers, crosslinking agents, and other variations known to those of ordinary skill in these arts may be incorporated into these materials.

A use of occlusive medical devices is related to Abdominal aortic aneurysms (AAA) and thoracic aortic aneurysms (TAA). Open surgery, primarily using clips or ligation techniques, has been the traditional means of treating AAAs and TAAs. Endovascular techniques, i.e. the placement of a stent graft at the site of the aneurysm, have become more popular. A material as described herein may be placed into n aneurysm to provide structure and assist in thrombosis to thereby coagulate the aneurysm to promote healing. For example, a material described herein can be extruded or molded in the shape of a fiber or coil, then dehydrated. The resulting dehydrated string or coil can be delivered via catheter to the site of a vascular malformation, such as an aneurysm, for vascular occlusion. The dehydrated material may be made to hydrate inside the blood vessel and/or to swell several times in size compared to its dehydrated state, while maintaining its original shape.

Chemoembolotherapy refers to the combination of providing mechanical blockage and localized, in situ delivery of chemotherapeutic agents. In the treatment of solid tumors, a therapeutic agent acts as an adjunct to the embolization. A clinical practice is mixing of therapeutic agents with embolic PVA particles for the delivery of drugs at tumor sites. This type of regional therapy may localize treatment at the site of the tumor, and therefore the therapeutic dose may be smaller than the effective systemic dose, reducing potential side effects and damage to healthy tissue. A material as described herein may be made a hydrated or dehydrated particles for use as embolization agents. A therapeutic agent may optionally be included in the particles to promote the particular use, e.g., a wound healing agent for wounds or a toxic chemical for chemotherapy.

Another application is tissue augmentation. Materials described herein can be used for augmentation of soft or hard tissue within the body of a patient. As such, they may be better than currently marketed collagen-based materials because they are less immunogenic and more persistent. Examples of soft tissue augmentation applications include sphincter (e.g., urinary, anal, esophageal) sphincter augmentation and the treatment of rhytids, wrinkles, and scars. Examples of hard tissue augmentation applications include the repair and/or replacement of bone and/or cartilaginous tissue.

Materials described herein may be adapted to make devices for use as a replacement material for synovial fluid in osteoarthritic joints, where the compositions serve to improve joint function by restoring a soft hydrogel network in the joint. The crosslinked polymer compositions can also be used as a replacement material for the nucleus pulposus of a damaged intervertebral disk. As such, the nucleus pulposus of the damaged disk is first removed, then the medical device, e.g., made of gellan, is injected or otherwise introduced into the center of the disk.

Adhesion prevention is another application. A medical device comprising a material described herein, e.g., gellan, is made with a suitable shape for deposition in the body after surgery is completed. For example, sheets and films are conventionally sued for adhesion prevention. Alternatively, powders or solutions of the gellan or other polysaccharides may be employed. In use, the devices are placed into the cavity after surgery is completed and before closure of the wound site.

The materials described herein may be made with a predetermined structure suitable for its intended use. A predetermined structure has a shape that is determined prior to introduction into a patient. For example, a polysaccharide hydrogel formed into a cylindrical or dog bone shape for plugging a void has a predetermined shape. In contrast, a polysaccharide sprayed onto a tissue or injected as a liquid into a tissue does not have a predetermined shape; instead, the materials are merely provided in any convenient form for delivery to the site. Thus, e.g., plugs, tampons, packing strips, sheets, particles, spheres, blocks, cubes, cylinders, and cones, are all contemplated as particular predetermined shapes. For example, packing made of a polysaccharide may be made for packing into a nasal or sinus cavity for treating patients that have undergone sinus surgeries. Or a stuffing may be made to fill a wound created surgically or by an accident. Or particles may be made to serve as a packing material, with large particles beings suitable for large wounds and microparticles being suited for smaller embolic applications or some minimally invasive surgeries requiring delivery by a catheter, e.g., with the microparticles having a maximum cross-sectional area of between about 1-10,000 square microns, e.g., a 100×100 micron cross-sectional area. Or, for example, strips provided, e.g., from a roll or other dispenser, with a thickness of between about 0.5 mm and about 5 mm may conveniently be used for packing a wound or lumen or void, e.g., a sinus cavity.

Coating of an implant is another application. A coating of a materials described herein, e.g, a polysaccharide, provides a biocompatible coating to reduce unwanted cellular and fibrous reaction to the coated implant. One method of application is to apply a solution of the coating material to the device, and to allow the coating to dry onto the implantable device.

Some materials and devices may advantageously be made to be triggerably degradable. Triggerably degradable means that exposure of the material or device to a triggering degradation agent will cause an accelerated degradation of the material relative to the rate of degradation of the material in the absence of the triggering agent. The triggering agent is a material that is not typically found in significant concentrations in the environment of the implant after it is implanted into the patient. Such materials may thus be removed at the convenience of the user when the material is no longer useful. Chelating agents are present in numerous over-the-counter eye, nasal and ear medications. For example, Disodium EDTA is such a preservative and chelating agent. In some embodiments, the triggered degradation is a result of exposure of the material to substantially deionized water. Substantially deionized water is water with no ions, or with a low concentration of ions, e.g., less than about 50 milliOsmoles, or less than about 10 milliOsmoles.

Other materials and devices may advantageously be made to have anisotropic swelling properties. For example, with respect to a cylindrical plug having a length and a diameter that is introduced into a needle track that has an opening and walls; the plug's diameter may be designed to swell to press against the walls of the track, while the length of the plug may be designed to swell to a different degree relative to the diameter, or to shrink.

Degradation of a material is a process that causes a material to lose its mechanical properties, e.g., its strength, cohesiveness, or resiliency. Degradation may occur by a variety of mechanisms, e.g., hydrolysis of chemical bonds, dissociation of ions that crosslink polymers that form the material, or a host-response to the material after its implantation into the host. In some instance, an implanted material is referred to as being dissolved, meaning that it has degraded to the point that the implanted material is essentially no longer visible at the implant site; such a process may occur by any of a variety of degradation mechanisms. Such dissolution may be modeled in a laboratory by maintaining a material in a container at physiological temperate, pH, and osmotic pressure until it is no longer visible to the naked eye.

Drug Delivery

Materials set forth herein may be associated with therapeutic agents, including drugs, imaging agents, diagnostic agents, prophylactic agents, and bioactive agents. A therapeutic agent may be mixed with a gel precursor that is in solution or disposed in a solvent, and the gel may be formed. Alternatively, the therapeutic agent may be introduced after the gel is formed or at an intermediate point in the gel formation process. Certain embodiments include gels that are made in a first solvent and exposed to a second solvent that contains the therapeutic agent so as to load the therapeutic agent into the gel.

Therapeutic agents include, for example, vasoactive agents, neuroactive agents, hormones, growth factors, cytokines, anesthetics, steroids, anticoagulants, anti-inflammatories, immunomodulating agents, cytotoxic agents, prophylactic agents, antibiotics, antivirals, antigens, and antibodies. Other therapeutic agents that can be provided in or on a coating material in accordance with the present invention include, but are not limited to, anti-thrombogenic agents such as heparin, heparin derivatives, urokinase, and PPack (dextrophenylalanine proline arginine chloromethylketone); anti-proliferative agents such as enoxaprin, angiopeptin, or monoclonal antibodies capable of blocking smooth muscle cell proliferation, hirudin, and acetylsalicylic acid; anti-inflammatory agents such as dexamethasone, prednisolone, corticosterone, budesonide, estrogen, sulfasalazine, and mesalamine; antineoplastic/antiproliferative/anti-mitotic agents such as paclitaxel, 5-fluorouracil, cisplatin, vinblastine, vincristine, epothilones, endostatin, angiostatin and thymidine kinase inhibitors; anesthetic agents such as lidocaine, bupivacaine, and ropivacaine; anti-coagulants such as D-Phe-Pro-Arg chloromethyl keton, an RGD peptide-containing compound, a polylysine-containing compound, heparin, antithrombin compounds, platelet receptor antagonists, anti-thrombin, anti-platelet receptor antibodies, aspirin, prostaglandin inhibitors, platelet inhibitors and tick antiplatelet peptides; vascular cell growth promoters such as growth factor inhibitors, growth factor receptor antagonists, transcriptional activators, and translational promoters; vascular cell growth inhibitors such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin; cholesterol-lowering agents; vasodilating agents; and agents which interfere with endogenous vasoactive mechanisms. Other examples of therapeutic agents include a radiophammaceutical, an analgesic drug, an anesthetic agent, an anorectic agent, an anti-anemia agent, an anti-asthma agent, an anti-diabetic agent, an antihistamine, an anti-inflammatory drug, an antibiotic drug, an antimuscarinic drug, an anti-neoplastic drug, an antiviral drug, a cardiovascular drug, a central nervous system stimulator, a central nervous system depressant, an anti-depressant, an anti-epileptic, an anxyolitic agent, a hypnotic agent, a sedative, an anti-psychotic drug, a beta blocker, a hemostatic agent, a hormone, a vasodilator, a vasoconstrictor, and a vitamin.

A gel may also include a second drug delivery device, e.g., microspheres, corticosteroids, neurotoxins, local anesthetics, opioid analgesics, vesicles, lipospheres, enzymes, combinations of these, and the like. Other therapeutic agents include, as listed in U.S. Pat. No. 6,342,250, incorporated by reference herein: Antidiarrhoeals such as diphenoxylate, loperamide and hyoscyamine; Antihypertensives such as hydralazine, minoxidil, captopril, enalapril, clonidine, prazosin, debrisoquine, diazoxide, guanethidine, methyldopa, reserpine, trimethaphan; Calcium channel blockers such as diltiazem, felodipine, amlodipine, nitrendipine, nifedipine and verapamil; Antiarrhyrthmics such as amiodarone, flecainide, disopyramide, procainamide, mexiletene and quinidine; Antiangina agents such as glyceryl trinitrate, erythrityl tetranitrate, pentaerythritol tetranitrate, mannitol hexanitrate, perhexilene, isosorbide dinitrate and nicorandil; Beta-adrenergic blocking agents such as alprenolol, atenolol, bupranolol, carteolol, labetalol, metoprolol, nadolol, nadoxolol, oxprenolol, pindolol, propranolol, sotalol, timolol and timolol maleate; Cardiotonic glycosides such as digoxin and other cardiac glycosides and theophylline derivatives; Adrenergic stimulants such as adrenaline, ephedrine, fenoterol, isoprenaline, orciprenaline, rimeterol, salbutamol, salmeterol, terbutaline, dobutamine, phenylephrine, phenylpropanolamine, pseudoephedrine and dopamine; Vasodilators such as cyclandelate, isoxsuprine, papaverine, dipyrimadole, isosorbide dinitrate, phentolamine, nicotinyl alcohol, co-dergocrine, nicotinic acid, glyceryl trinitrate, pentaerythritol tetranitrate and xanthinol; Antimigraine preparations such as ergotamine, dihydroergotamine, methysergide, pizotifen and sumatriptan; Anticoagulants and thrombolytic agents such as warfarin, dicoumarol, low molecular weight heparins such as enoxaparin, streptokinase and its active derivatives; Hemostatic agents such as aprotinin, tranexamic acid and protamine; Analgesics and antipyretics including the opiold analgesics such as buprenorphine, dextromoramide, dextropropoxyphene, fentanyl, alfentanil, sufentanil, hydromorphone, methadone, morphine, oxycodone, papaveretum, pentazocine, pethidine, phenoperidine, codeine dihydrocodeine, acetylsalicylic acid (aspirin), paracetamol, and phenazone; Neurotoxins such as capsaicin; Hypnotics and sedatives such as the barbiturates amylobarbitone, butobarbitone and pentobarbitone and other hypnotics and sedatives such as chloral hydrate, chlormethiazole, hydroxyzine and meprobamate; Antianxiety agents such as the benzodiazepines alprazolam, bromazepam, chlordiazepoxide, clobazam, chlorazepate, diazepam, flunitrazepam, flurazepam, lorazepam, nitrazepam, oxazepam, temazepam and triazolam; Neuroleptic and antipsychotic drugs such as the phenothiazines, chlorpromazine, fluphenazine, pericyazine, perphenazine, promazine, thiopropazate, thioridazine, trifluoperazine; and butyrophenone, droperidol and haloperidol; and other antipsychotic drugs such as pimozide, thiothixene and lithium; Antidepressants such as the tricyclic antidepressants amitryptyline, clomipramine, desipramine, dothiepin, doxepin, imipramine, nortriptyline, opipramol, protriptyline and trimipramine and the tetracyclic antidepressants such as mianserin and the monoamine oxidase inhibitors such as isocarboxazid, phenelizine, tranylcypromine and moclobemide and selective serotonin re-uptake inhibitors such as fluoxetine, paroxetine, citalopram, fluvoxamine and sertraline; CNS stimulants such as caffeine and 3-(2-aminobutyl) indole; Anti-alzheimer's agents such as tacrine; Anti-Parkinson's agents such as amantadine, benserazide, carbidopa, levodopa, benztropine, biperiden, benzhexol, procyclidine and dopamine-2 agonists such as S(−)-2-(N-propyl-N-2-thienylethylamino)-5-hydroxytetralin (N-0923); Anticonvulsants such as phenyloin, valproic acid, primidone, phenobarbitone, methylphenobarbitone and carbamazepine, ethosuximide, methsuximide, phensuximide, sulthiame and clonazepam; Antiemetics and antinauseants such as the phenothiazines prochloperazine, thiethylperazine and 5HT-3 receptor antagonists such as ondansetron and granisetron, as well as dimenhydrinate, diphenhydramine, metoclopramide, domperidone, hyoscine, hyoscine hydrobromide, hyoscine hydrochloride, clebopride and brompride; Non-steroidal anti-inflammatory agents including their racemic mixtures or individual enantiomers where applicable, preferably which can be formulated in combination with dermal penetration enhancers, such as ibuprofen, flurbiprofen, ketoprofen, aclofenac, diclofenac, aloxiprin, aproxen, aspirin, diflunisal, fenoprofen, indomethacin, mefenamic acid, naproxen, phenylbutazone, piroxicam, salicylamide, salicylic acid, sulindac, desoxysulindac, tenoxicam, tramadol, ketoralac, flufenisal, salsalate, triethanolamine salicylate, aminopyrine, antipyrine, oxyphenbutazone, apazone, cintazone, flufenamic acid, clonixeril, clonixin, meclofenamic acid, flunixin, coichicine, demecolcine, allopurinol, oxypurinol, benzydamine hydrochloride, dimefadane, indoxole, intrazole, mimbane hydrochloride, paranylene hydrochloride, tetrydamine, benzindopyrine hydrochloride, fluprofen, ibufenac, naproxol, fenbufen, cinchophen, diflumidone sodium, fenamole, flutiazin, metazamide, letimide hydrochloride, nexeridine hydrochloride, octazamide, molinazole, neocinchophen, nimazole, proxazole citrate, tesicam, tesimide, tolmetin, and triflumidate; Antirheumatoid agents such as penicillamine, aurothioglucose, sodium aurothiomalate, methotrexate and auranofin; Muscle relaxants such as baclofen, diazepam, cyclobenzaprine hydrochloride, dantrolene, methocarbamol, orphenadrine and quinine; Agents used in gout and hyperuricaemia such as allopurinol, colchicine, probenecid and sulphinpyrazone; Oestrogens such as oestradiol, oestriol, oestrone, ethinyloestradiol, mestranol, stilboestrol, dienoestrol, epioestriol, estropipate and zeranol; Progesterone and other progestagens such as allyloestrenol, dydrgesterone, lynoestrenol, norgestrel, norethyndrel, norethisterone, norethisterone acetate, gestodene, levonorgestrel, medroxyprogesterone and megestrol; Antiandrogens such as cyproterone acetate and danazol; Antioestrogens such as tamoxifen and epitiostanol and the aromatase inhibitors, exemestane and 4-hydroxy-androstenedione and its derivatives; Androgens and anabolic agents such as testosterone, methyltestosterone, clostebol acetate, drostanolone, furazabol, nandrolone oxandrolone, stanozolol, trenbolone acetate, dihydro-testosterone, 17-.alpha.-methyl-19-nortestosterone and fluoxymesterone; 5-alpha reductase inhibitors such as finasteride, turosteride, LY-191704 and MK-306; Corticosteroids such as betamethasone, betamethasone valerate, cortisone, dexamethasone, dexamethasone 21-phosphate, fludrocortisone, flumethasone, fluocinonide, fluocinonide desonide, fluocinolone, fluocinolone acetonide, fluocortolone, halcinonide, halopredone, hydrocortisone, hydrocortisone 17-valerate, hydrocortisone 17-butyrate, hydrocortisone 21-acetate, methylprednisolone, prednisolone, prednisolone 21-phosphate, prednisone, triamcinolone, triamcinolone acetonide; Further examples of steroidal antiinflammatory agents such as cortodoxone, fludroracetonide, fludrocortisone, difluorsone diacetate, flurandrenolone acetonide, medrysone, amcinafel, amcinafide, betamethasone and its other esters, chloroprednisone, clorcortelone, descinolone, desonide, dichlorisone, difluprednate, flucloronide, flumethasone, flunisolide, flucortolone, fluoromethalone, fluperolone, fluprednisolone, meprednisone, methylmeprednisolone, paramethasone, cortisone acetate, hydrocortisone cyclopentylpropionate, cortodoxone, flucetonide, fludrocortisone acetate, flurandrenolone acetonide, medrysone, aincinafal, amcinafide, betamethasone, betamethasone benzoate, chloroprednisone acetate, clocortolone acetate, descinolone acetonide, desoximetasone, dichlorisone acetate, difluprednate, flucloronide, flumethasone pivalate, flunisolide acetate, fluperolone acetate, fluprednisolone valerate, paramethasone acetate, prednisolamate, prednival, triamcinolone hexacetonide, cortivazol, formocortal and nivazol; Pituitary hormones and their active derivatives or analogs such as corticotrophin, thyrotropin, follicle stimulating hormone (FSH), luteinising hormone (LH) and gonadotrophin releasing hormone (GnRH); Hypoglycemic agents such as insulin, chlorpropamide, glibenclamide, gliclazide, glipizide, tolazamide, tolbutamide and metformin; Thyroid hormones such as calcitonin, thyroxine and liothyronine and antithyroid agents such as carbimazole and propylthiouracil; Other miscellaneous hormone agents such as octreotide; Pituitary inhibitors such as bromocriptine; Ovulation inducers such as clomiphene; Diuretics such as the thiazides, related diuretics and loop diuretics, bendrofluazide, chlorothiazide, chlorthalidone, dopamine, cyclopenthiazide, hydrochlorothiazide, indapamide, mefruside, methycholthiazide, metolazone, quinethazone, bumetanide, ethacrynic acid and frusemide and potasium sparing diuretics, spironolactone, amiloride and triamterene; Antidiuretics such as desmopressin, lypressin and vasopressin including their active derivatives or analogs; Obstetric drugs including agents acting on the uterus such as ergometrine, oxytocin and gemeprost; Prostaglandins such as alprostadil (PGE1), prostacyclin (PGI2), dinoprost (prostaglandin F2-alpha) and misoprostol; Antimicrobials including the cephalosporins such as cephalexin, cefoxytin and cephalothin; Penicillins such as amoxycillin, amoxycillin with clavulanic acid, ampicillin, bacampicillin, benzathine penicillin, benzylpenicillin, carbenicillin, cloxacillin, methicillin, phenethicillin, phenoxymethylpenicillin, flucloxacillin, meziocillin, piperacillin, ticarcillin and azlocillin; Tetracyclines such as minocycline, chlortetracycline, tetracycline, demeclocycline, doxycycline, methacycline and oxytetracycline and other tetracycline-type antibiotics; Aminoglycosides such as amikacin, gentamicin, kanamycin, neomycin, netilmicin and tobramycin; Antifungals such as amorolfine, isoconazole, clotrimazole, econazole, miconazole, nystatin, terbinafine, bifonazole, amphotericin, griseofulvin, ketoconazole, fluconazole and flucytosine, salicylic acid, fezatione, ticlatone, tolnaftate, triacetin, zinc, pyrithione and sodium pyrithione; Quinolones such as nalidixic acid, cinoxacin, ciprofloxacin, enoxacin and norfloxacin; Sulphonamides such as phthalysulphthiazole, sulfadoxine, sulphadiazine, sulphamethizole and sulphamethoxazole; Sulphones such as dapsone; Other miscellaneous antibiotics such as chloramphenicol, clindamycin, erythromycin, erythromycin ethyl carbonate, erythromycin estolate, erythromycin glucepate, erythromycin ethylsuccinate, erythromycin lactobionate, roxithromycin, lincomycin, natamycin, nitrofurantoin, spectinomycin, vancomycin, aztreonain, colistin IV, metronidazole, tinidazole, fusidic acid, trimethoprim, and 2-thiopyridine N-oxide; halogen compounds, particularly iodine and iodine compounds such as iodine-PVP complex and diiodohydroxyquin, hexachlorophene; chlorhexidine; chloroamine compounds; and benzoylperoxide; Antituberculosis drugs such as ethambutol, isoniazid, pyrazinamide, rifampicin and clofazimine; Antimalarials such as primaquine, pyrimethamine, chloroquine, hydroxychloroquine, quinine, mefloquine and halofantrine; Antiviral agents such as acyclovir and acyclovir prodrugs, famcyclovir, zidovudine, didanosine, stavudine, lamivudine, zalcitabine, saquinavir, indinavir, ritonavir, n-docosanol, tromantadine and idoxuridine; Anthelmintics such as mebendazole, thiabendazole, niclosamide, praziquantel, pyrantel embonate and diethylcarbamazine; Cytotoxic agents such as plicainycin, cyclophosphamide, dacarbazine, fluorouracil and its prodrugs (described, for example, in International Journal of Pharmaceutics 111, 223-233 (1994)), methotrexate, procarbazine, 6-mercaptopurine and mucophenolic acid; Anorectic and weight reducing agents including dexfenfluramine, fenfluramine, diethylpropion, mazindol and phentermine; Agents used in hypercalcaemia such as calcitriol, dihydrotachysterol and their active derivatives or analogs; Antitussives such as ethylmorphine, dextromethorphan and pholcodine; Expectorants such as carbolcysteine, bromhexine, emetine, quanifesin, ipecacuanha and saponins; Decongestants such as phenylephrine, phenylpropanolamine and pseudoephedrine; Bronchospasm relaxants such as ephedrine, fenoterol, orciprenaline, rimiterol, salbutamol, sodium cromoglycate, cromoglycic acid and its prodrugs (described, for example, in International Journal of Pharmaceutics 7, 63-75 (1980)), terbutaline, ipratropium bromide, salmeterol and theophylline and theophylline derivatives; Antihistamines such as meclozine, cyclizine, chlorcyclizine, hydroxyzine, brompheniramine, chlorpheniramine, clemastine, cyproheptadine, dexchlorpheniramine, diphenhydramine, diphenylamine, doxylamine, mebhydrolin, pheniramine, tripolidine, azatadine, diphenylpyraline, methdilazine, terfenadine, astemizole, loratidine and cetirizine; Local anaesthetics such as bupivacaine, amethocaine, lignocaine, lidocaine, cinchocaine, dibucaine, mepivacaine, prilocaine, etidocaine, veratridine (specific c-fiber blocker) and procaine; Stratum corneum lipids, such as ceramides, cholesterol and free fatty acids, for improved skin barrier repair [Man, et al. J. Invest. Dermatol., 106(5), 1096, (1996)]; Neuromuscular blocking agents such as suxamethonium, alcuronium, pancuronium, atracurium, gallamine, tubocurarine and vecuronium; Smoking cessation agents such as nicotine, bupropion and ibogaine; Insecticides and other pesticides which are suitable for local application; Dermatological agents, such as vitamins A, C, B₁, B₂, B₆, B₁₂ₐ and E, vitamin E acetate and vitamin E sorbate; Allergens for desensitization such as house, dust or mite allergens; Nutritional agents, such as vitamins, essential amino acids and fats; Keratolytics such as the alpha-hydroxy acids, glycolic acid and salicylic acid.

Additional therapeutic agents include anti-glaucoma drugs, e.g., timolol, dorzolamide hydrochloride, latanoprost, and brimonidine (see also: 1998 Physicians' Desk Reference for Ophthalmology). Other agents are ones having neuroprotective properties for ganglion cells and/or optic nerve axons in glaucoma, as well as gene delivery to ocular tissues. Additional therapeutic agents include antifungal agents, antibiotic agents, for treating keratitis, agents for treating endophthalmitis, anti-inflammatory medications, and steroids. Additional therapeutic agents include those for antimicrobial therapy, antiviral agents for herpes simplex, zoster keratitis, and cytomegalovirus retinitis.

Persons of skill in these arts, after reading this disclosure, will be able to use a variety of techniques to incorporate therapeutic agents into materials described herein. For example, polar agents can be added to an acid gellan-dimethyl sulfoxide (DMSO) solution and coextruded. The high polarity of DMSO allows the solution or suspension of the polar agent. Similarly, other polar solvents that are compatible with gellan may be used. Alternatively, water-soluble agents can be successfully incorporated by extrusion into mixed organic solvent-water systems such as 70% methyl, ethyl or isopropyl alcohols or 70% acetone. The solvent mixtures are compatible with both the polymers and the agents so that they maybe readily combined. Alternatively, non-polar agents that have poor solubility in water or polar solvents can be incorporated into the extrusion mixture by formation of emulsions or encapsulation in particles. Such techniques are known to persons of ordinary skill in these arts.

Another method of delivery involves exposing a material to DMSO or Methyl-sulfonyl-methane (MSM), with a therapeutic agent being contained therein. The implant, with the DMSO, MSM, or other suitable solvent still present, may be implanted. The DMSO, MSM, and/or other solvent, enhances delivery of the drug into a tissue.

The principles set forth in the context of gellan may be applied to polysaccharides and polysaccharide-like materials. In general, a polysaccharide may be dissolved in DMSO or similar polar organic solvents, after neutralization of its charges by association with a salt, e.g., by making them a free base or a free acid. Then more organic solvent may be used to introduce an agent, or other solvents may be introduced to create a mixture that is compatible with both the desired agent and the polysaccharide.

A variety of materials and materials processing techniques are set forth herein. Persons of skill in these arts, after reading this disclosure, will be able to use a variety of techniques in combination with such materials and methods. An agent may potentially be associated with a material or device at a variety of stages, including the manufacture of the material, or after the material is formed into the implant. During manufacture of the material, an agent and the material components may be combined in solvents that are suitable for both. Alternatively, a material may first be formed and then subsequently swelled in a solvent that contains the agent; after removal of the solvent, the material may be dried or put into a different solvent to deswell the material. Alternatively, the materials may be made so as to physically entrap the agents. Alternatively, emulsion techniques may be used to introduce the agents into the materials.

Antimicrobial Agents and Preservatives

Gels and other materials and devices set forth herein may optionally contain antimicrobial agents and/or preservatives to prevent growth of microorganisms. The gel would entrap such agents or preservatives at the site where the gel is formed in a patient, or could slowly elute such agents or preservatives into the patient, e.g., into the bloodstream or other tissues. Various agents are described in priority document U.S. Provisional Application No. 60/550,132, entitled “Punctum Plugs, Materials, And Devices”, and may be combined with the gels and devices described herein.

Colloidal or particulate silver is another agent that may be used in these gels and devices. Colloidal or particulate silver exists in an aggregated or crystalline state and is essentially uncharged. Colloidal or particulate silver does not interact with charged groups on polysaccharides because it does not carry a charge; as a result, colloidal or particulate silver can not be a crosslinking ion that crosslinks a polysaccharide.

Inclusion of large particles such as silver powder has the effect of diminishing extrusion viscosity and fiber strength. In the case of metallic silver and silver salt nanoparticles, this can be overcome by particle precipitation as extrusions are gelled. For example, if a silver nitrate/acidified gellan gum/DMSO solution is extruded into a coagulation bath consisting of sodium chloride, very fine particles of insoluble silver chloride will precipitate within the extrusion as gelation occurs. Likewise metallic silver will be precipitated upon contact with a coagulation bath consisting of reducing agents such as ascorbic acid, hydroquinone, ferrous salts or cuprous salts.

For the production of metallic silver nanoparticles using this method, it is advantageous to extrude a silver nitrate/acidified gellan gum/DMSO solution into a coagulation bath containing ascorbic acid. Extrusions turn from clear to yellow upon contact with the bath, indicating the formation of silver nanoparticles. After neutralization, stretching and dehydrating extrusions possessed enough strength to be easily stretched to over twice their original length.

Silver nanoparticle-containing gels made using the ascorbate reduction method were found to lose color if placed into physiological saline but not if placed into water. Without being committed to a specific mechanism of action, it is thought that chloride ions present in physiological saline induce loss of silver from the surface of nanoparticles. Subsequently silver chloride is formed. Silver chloride is slightly soluble in water and therefore leaches out over a 2-3 week period. Leaching of silver ions is important for ensuring proper function of antimicrobial properties.

Another preservative commonly used with polysaccharide materials is boric acid and its salts. A number of polymers—polyvinyl alcohol, guar gum and locust bean gum—form gel-like materials upon esterification through reaction with boric acid or its salts. This is possible due to the presence of 1,2 cis-diol groups present in these polymers. Gellan gum possesses 1,2 cis-diol groups on rhamonpyranosyl residues present in the polymer chain. It is herein disclosed that gellan gum is capable of reacting with boric acid and its salts to create gels whose properties are pH-dependent. The pH to which gellan borate gels would be exposed in medical applications would typically be slightly alkaline (e.g., about pH=7.4). Under these conditions borate esters are stable. The presence of borate bound within gellan gum gels should inhibit growth of microorganisms such as bacteria and fungi. Properties such as saline gelation and water solubility remain unaffected. Unlike other gels formed by borate esterification, gellan borate gels are rigid and do not easily flow when subjected to pH ranges normally encountered in the body.

The antimicrobial agent or preservative may be mixed with a solvent that is used to dissolve or suspend the polysaccharide; an advantage of this process is that the agent or preservative is dispersed through the solvent and is relatively well mixed into the final composition. Or the agent or preservative may be introduced into a powder of the polysaccharide. The agent or preservative may also be introduced at other points of processing, with the choice depending on the type of agent, solvents, and eventual application.

For example, triclosan, a common antimicrobial agent, is insoluble in water but is highly soluble in DMSO and alcohols. Triclosan was added to a 15% acid gellan-DMSO solution to make a mixture of 0.5% triclosan and 15% acid gellan. The mixture was deaerated under vacuum for 2 hours to remove air bubbles and extruded under 45 psi air pressure into a coagulation bath of 2.5% sodium bicarbonate-7.5% sodium chloride. Extrusions were washed briefly in water chilled to 1-2° C. and then allowed to air dry under tension. In contrast to clear extrusions made from gellan alone, those containing triclosan appeared white. If soaked in 70% isopropyl alcohol, extrusions became clear, indicating elution of triclosan.

Another method of delivery involves exposing a material to DMSO or Methyl-sulfonyl-methane (MSM), with a therapeutic agent being contained therein. The implant, with the DMSO, MSM, or other suitable solvent still present, may be implanted. The DMSO, MSM, and/or other solvent, enhances delivery of the drug into a tissue.

And, for example, Gellan gum was acidified by washing three times with 5% citric acid in water. Resulting acidified gellan powder was subsequently rinsed with water and alcohol and allowed to dry. Acidified powder (15 grams) of gellan gum was dissolved into 99 milliliters of dimethyl sulfoxide. A silver solution was then made by dissolution of 0.157 grams of silver nitrate in DMSO. One milliliter of this solution was added to the 99 milliliters of gellan gum solution and was subjected to a vacuum to remove air bubbles. This solution was extruded under air pressure (45-50 pounds per square inch) into 10% ascorbic acid in water and allowed to incubate for 30 minutes, at which time extrusions changed from clear and colorless to a light straw color. They were subsequently washed three times in distilled water to remove any free ions, unbound silver particles and ascorbic acid. After dehydration through a graded ethanol series extrusions were stretched to twice their original length and allowed to dry.

Occlusive devices were then fabricated by cutting neutralized extrusions into cylindrical pieces. Their dry dimensions were 1.524 millimeters in length and 0.254 millimeters in diameter. Once placed into physiological saline and allowed to swell to their maximum extent, they had dimensions of 1.27 millimeters in length and 1.016 millimeters in diameter. After one week in the physiological saline solution they began to lose color and after 2-3 weeks they became clear.

As another example, gellan gum was acidified by washing three times with 5% citric acid in water. Resulting acidified gellan powder was subsequently rinsed with water and alcohol and allowed to dry. Acidified powder (15 grams) of gellan gum was dissolved into 99 milliliters of dimethyl sulfoxide. A silver solution was then made by dissolution of 0.157 grams of silver nitrate in DMSO. One milliliter of this solution was added to the 99 milliliters of gellan gum solution and was subjected to a vacuum to remove air bubbles. This solution was extruded under air pressure (45-50 pounds per square inch) into 10% ascorbic acid in water and allowed to incubate for 30 minutes, at which time extrusions changed from clear and colorless to a light straw color. They were subsequently washed three times in distilled water to remove any free ions, unbound silver particles and ascorbic acid. After dehydration through a graded ethanol series extrusions were stretched to twice their original length and allowed to dry.

After drying, extrusions were placed into a 5% solution of calcium chloride in 70% aqueous ethanol and allowed to incubate for 2 hours. After rinsing in 70% aqueous ethanol for two hours and dehydration in 91% ethanol, extrusions were allowed to air dry. Occlusive devices were then fabricated by cutting calcium gellan extrusions into cylindrical pieces. Their dry dimensions were 1.524 millimeters in length and 0.254 millimeters in diameter. Once placed into physiological saline and allowed to swell to their maximum extent, they had dimensions of 1.27 millimeters in length and 0.575 millimeters in diameter. After 2-3 weeks in the distilled water they retained their original straw color.

As another example, gellan gum was acidified by washing three times with 5% citric acid in water. Resulting acidified gellan powder was subsequently rinsed with water and alcohol and allowed to dry. Acidified powder (15 grams) of gellan gum was dissolved into 100 milliliters of dimethyl sulfoxide to make a 15% solution which was subjected to a vacuum to remove air bubbles. This solution was extruded under air pressure (45-50 pounds per square inch) into 10% sodium citrate in water and allowed to incubate for 30 minutes. It was subsequently washed in 1.0% sodium chloride to remove any excess citrate ions. Extrusions were then placed into a 5% solution of sodium tetraborate decahydrate and incubated for 2 hours. After washing in 1% sodium chloride and dehydration through a graded ethanol series, extrusions were stretched. Upon stretching it was found that extrusions demonstrated acceptable strength so long as they were not deformed beyond their elastic limit. Plastic deformation of extrusions was not possible due to breakage.

As another example, gellan gum was acidified by washing three times with 5% citric acid in water. Resulting acidified gellan powder was subsequently rinsed with water and alcohol and allowed to dry. Acidified powder (15 grams) of gellan gum was dissolved into 100 milliliters of dimethyl sulfoxide to make a 15% solution which was subjected to a vacuum to remove air bubbles. This solution was extruded under air pressure (45-50 pounds per square inch) into 10% sodium citrate in water and allowed to incubate for 30 minutes. It was subsequently washed in 1.0% sodium chloride to remove any excess citrate ions. Extrusions were dehydrated in a graded alcohol series to 91% alcohol and subsequently stretched to twice their original length. They were allowed to air dry.

Upon drying extrusions were placed into a saturated solution of sodium tetraborate decahydrate in 70% aqueous methanol. Incubation in this medium lasted for two hours, followed by a two hour rinse in 70% methanol and 100% methanol. Extrusions remained very strong even esterification with borate.

Additional Embodiments

An embodiment is a medical implant comprising a hydrogel comprised of a gellan welan, S-88, S-198 or rhamsan gum polymer capable of forming a hydrogel in the presence of bodily fluids. Such hydrogels may have sufficient strength to serve as a device or a component of a device including temporary occlusive devices such as a packing or plug. Such hydrogel may be stable for, e.g., less than 5, 10, 15, 30, 60, 120 days, up to one year, up to two years, up to five years. The gellan may be, e.g., an organic or inorganic salt of gellan, and/or welan, and/or S-88, and/or S-198 and/or rhamsan gums. Such materials or implants may be depolymerized to facilitate degradation. Such material or implant may be treated with oxidizing agents including, but not limited to, periodic acid, salts of periodic acid, hydrogen peroxide, benzoyl peroxide, etc. Alternatively, such material or implant may be converted to free acid form and/or heated to, e.g., effect acid catalyzed hydrolysis of the molecules. Such materials or implants may be, e.g, processed at ambient temperature from a solvent that comprises at least one of: water, organic solvent, a ketone organic solvent, a sulfoxide organic solvent, or other polar aprotic solvents. Some embodiments describe shaping of a device and subsequent depolymerization of the polymers contained therein. Such materials or implants may be capable of swelling 100% or more from a dry state when exposed to bodily fluids. Such materials or implants may be used to deliver a therapeutic agent, e.g., antimicrobial agents, medicaments, biologicals and/or living cells to the site of implantation.

Other embodiments are devices or materials are related to water-soluble polymers which gel under physiological conditions, e.g., in the presence of excess cations. Such polymer may be, e.g., gellan gum, whose duration in the body is dependent primarily on its degree of polymerization. A method of removing certain embodiments of these materials is the application of ion-free water. In some embodiments, the material or device swells in bodily fluid by at least 80% compared to its dimensions when dry. The water solubility of the polymer, e.g., gellan, may be regulated by the addition of another polymer capable of hydrogen bonding to itself or to gellan gum; for example, polyvinyl alcohol or tamarind seed gum and/or their derivatives. Some embodiments include hydrogels for delivering antimicrobial agents, medicaments, biologicals and/or living cells to the site of implantation. Further embodiments include methods for adding preservatives to hydrogel devices by esterification with borate or by precipitation/reduction of silver salts.

Other embodiments are devices or materials that are not true liquids, but are instead fluids composed of submicron particles of gel material suspended in water. Methods of making such devices or materials may include providing a polymer such as gellan gum, curdlan, or agarose, and making it into a dispersion in cold water, which may then be heated to make a flowable composition. An embodiment is a degradable material or medical implant comprising sufficient strength to serve as an occlusive material, e.g., a plug or packing. Such a material or implant may comprise polymers which are soluble or dispersable in hot water but which form gels upon cooling. Preparations to promote gel fluidity may be achieved by mechanical disruption of a cooling polymer solution, as by agitation, stirring, homogenization, ultrasonic disruption, etc. Such materials or devices may, when disrupted, be concentrated by evaporation, filtering, centrifugation. Such materials or devices may be processed from concentrated, fluid gels by, e.g., molding, extrusion, or casting. Such materials or devices may be prepared so as to be capable of being dried but, upon rehydration become a viscous fluid. Such materials or devices may be used to deliver a therapeutic agent.

Other embodiments are devices or materials that include an ionic hydrogel comprising a mineral-forming substance. Such materials or devices may include an organic phase of one or more anionic polymers crosslinked to an inorganic phase of an insoluble metal salt having sufficient strength to serve as an occlusive device such as a packing or plug. Such devices and materials may include, e.g., gellan, alginates, poly(acrylic acid), xanthan, carrageenan, carboxymethyl cellulose, carboxymethyl chitosan, hydroxypropyl carboxymethyl cellulose, pectin, welan, gum Arabic, karaya gum, psyllium seed gum, carboxymethyl guar, and mesquite gum. Metal salts may include a metal ion having a charge equal to or greater than +2. An inorganic constituent of the hydrogel may be, e.g., a metal silicate, hydroxide, phosphate, carbonate, chromate, sulfate, or vanadate. The organic and inorganic constituents of the hydrogel may be ionically crosslinked by metal ions. Methods of using such hydrogels may include exposure to chelating agents and achieving a dissolution rate that is slower than the same hydrogel crosslinked by metal ions alone. Some of such hydrogels are capable of swelling 100% or more from a dry state when exposed to bodily fluids. These hydrogels may be used to deliver therapeutic agents.

Other embodiments are devices or materials related to covalently crosslinked ionic hydrogels and uses thereof, e.g., as reversible occlusive materials. Certain embodiments include oxidation-sensitive hydrogels having covalently crosslinked polymers which are capable of binding metal ions, especially those of transition metals. Uses include, e.g., an occlusive hydrogel material such as a plug or packing. Some embodiments include at least one polymer is capable of binding metals through formation of coordination compounds or ionic bonds. Some embodiments include polymers, e.g., alginate, gellan, poly(acrylic acid), chitin, chitosan, oxidized cellulose, carboxymethyl cellulose, xanthan, carrageenan, pectin, hydroxypropyl carboxymethyl cellulose, welan gum, cellulose phosphate, or croscarmellose sodium. Some embodiments contain polymers incapable of binding metals but which participate in covalent crosslinking. Some embodiments include polymers covalently crosslinked in such a manner that metal binding capacity is partially or fully maintained. Some polymers may, furthermore, have anionic, cationic and/or hydroxyl functional groups. Some embodiments include polymers crosslinked by covalent modification of hydroxyl groups. Some embodiments include polymers crosslinked by reaction of hydroxyl groups with crosslinking agents such as epihalohydrins, dialdehydes, citric acid, butanetetracarboxylic acid, or polymaleic anhydride. Some embodiments include polymers crosslinked to themselves or another polymer by reaction of hydroxyl groups with acidified carboxymethyl groups. Some embodiments include polymers that dissolve and/or disintegrate upon exposure to the combination of oxidizing agents and catalytic metal ions. Some embodiments include hydrogels for delivering antimicrobial agents, medicaments, biologicals and/or living cells to the site of implantation.

Other embodiments are devices or materials related to the situ removal of hydrogel devices by oxidative degradation. Certain embodiments include removing hydrogel medical devices through oxidative-reductive reactions involving oxidizing agents and metal ion catalysts. Such hydrogels may be sensitive to oxidative degradation, e.g., by free radicals. Certain embodiments are capable of binding metal ion catalysts. Examples of such metal ions are heavy metal or transition metal ions, ferrous, ferric, cuprous and cupric ions. Examples of oxidizing agents include phenols and phenolic compounds, benzoyl peroxide, hydrogen peroxide, ascorbate, and so forth. In some embodiments, a hydrogel either contains or first binds catalytic metal ions and then oxidizes once exposed to appropriate oxidizing agents.

Other embodiments are devices or materials related to removal of hydrogel occlusive devices by changes in tonicity. For example, a method for safe removal of hydrogel medical devices involves using hypertonic solutions of water soluble polymers and/or inorganic salts to decrease their dimensions. Alternatively, biocompatible liquid polymers such as polyethylene glycol-200 (PEG-200) could be utilized in place of aqueous solutions. Some embodiments can be dissolved to make at least a 25% solution in physiological saline without undue increases in viscosity. In some cases, a hypertonic solution removes water from a hydrogel device to thereby ease its removal. Similarly, such hydrogel devices may be made to shrink in dimensions upon removal of water. Certain embodiments are related to methods of removing an implant following changes in dimensions of the hydrogel.

Other embodiments are devices or materials related to anisotropic hydrogel materials. In some embodiments, these are used as occlusive devices. One embodiment is an occlusive device or material that is a dried hydrogel material which, upon exposure to water, saline or bodily fluids, swells to different extents in at least one of three dimensions or shrinks in at least one of three dimensions. Methods of producing an anisotropic structure and/or molecular orientation may include stretching, deforming, spray coating, spin coating, ordered convection, or directional gelling or freezing. In some embodiments, the hydrogel material has sufficient strength to serve as an occlusive device such as a plug or packing. In some embodiments, the material has sufficient strength to serve as a suturing material which tightens during hydration.

Swellable Temporary Punctum Plugs

A series of swellable temporary punctum plugs have been made that embody many of the inventions described herein. A swellable temporary punctum plug may be designed to sit beyond the punctal ring, and can be removed in one of several ways. It may be irrigated with saline solution, it can be palpated after hydration to break the plug into pieces so it can be passed through the lacrimal system or upward through the punctum, it can be probed out with a lacrimal probe, or it may be left in place to dissolve, e.g., within 30 days of insertion. The swellable temporary punctum plug may be designed to completely dissolve within 30 days, and move out of the lacrimal system via the nasolacrimal duct. It is then expelled through the nasal cavity or into the stomach where it is ingested and passed through the excretory system. Swellable temporary punctum plugs can be made to have no sharp edges after they are hydrated, with the shape of the plug conforming to the volume that constrains it. This feature serves to limit any foreign body reaction, and the short duration serves to limit any infection that may occur. Swellable temporary punctum plugs have been made that generally take 5-10 minutes to become fully hydrated by the action of tear production, or by the use of saline drops if tear volume is not sufficient (as may be expected from patients suffering from dry eye).

### REFERENCES

Certain references conveniently provide additional information about certain aspects of making and using the invention. These are provided herein by way of reference to the reader.
Buettner, G R; Jurkiewicz, B A. Catalytic metals, ascorbate and free radicals: combinations to avoid. Radiation Research. 145(5):532-41, May 1996.
Callegaro, L.; Biviano, F.; Crescenzi, V. Auto-cross-linked gellan gum. U.S. Pat. No. 6,172,219, Jan. 9, 2001.
Doyle, P. J.; Harvey, Wilson; Stilwell, Reginald L. Bioresorbable alginate derivatives. U.S. Pat. No. 6,440,940, Aug. 27, 2002.
Klimiuk, E; Kuczajowska-Zadrozna, M. The Effect of Poly(Vinyl Alcohol) on Cadmium Adsorption and Desorption from Alginate Adsorbents. Polish Journal of Environmental Studies. 11(4): 375-384, 2002.
Lazaro, N.; Sevilla, A L; Morales, S; Marques, A M. Heavy metal biosorption by gellan gum gel beads. Water Research. 37(9):2118-26, May 2003.
Luzio, Gary A. Thompson, Samuel A. Medical devices subject to triggered disintegration. U.S. Pat. No. 5,531,716, Jul. 2, 1996.
Marder, Herman L.; Field, Nathan D.; Shinohara, Makoto. Preparation of water-insoluble carboxymethyl cellulose absorbents. U.S. Pat. No. 4,200,737, Apr. 29, 1980.
Mooney; David J.; Bouhadir; Kamal H.; Wong; Wai Kung; Rowley; Jon A. Polymers containing polysaccharides such as alginates or modified alginates. U.S. Pat. No. 6,642,363, Nov. 4, 2003.
Nicolay, X.; Therasse, Ph.; De Maesschalck, E.; Simon, J-P. Cell Entrapment and Coated-System Immobilization (CESCI Technology) for the Biological Treatment of Industrial Gaseous Emmissions. Illkirch, France: XIth International Workshop on Bioencapsulation, May 25-27, 2003.
Pattanapipitpaisal, P; Brown, N. L.; Macaskie, L. E. Chromate reduction by Microbacterium liquefaciens immobilized in polyvinyl alcohol. Biotechnology Letters. 23:61-65, 2001.
Reid, Albert R. Water-soluble polymers and process of preparing. U.S. Pat. No. 3,379,720, Apr. 23, 1968.
Schoggen, Howard L. Slurry process for the production of absorptive carboxy methyl cellulose fibers. U.S. Pat. No. 3,678,031, Jul. 18, 1972.
Shinohara, Makoto; Field, Nathan D. Preparation of water-insoluble carboxymethyl cellulose absorbents. U.S. Pat. No. 4,200,736, Apr. 29, 1980.

All patents, patent applications, references, and publications herein are hereby incorporated by reference herein.

1. A swellable medical device that swells after introduction into a patient to occlude a lumen or void defined by a tissue, the device comprising: a predetermined structure comprising a biocompatible hydrogel comprising at least one polysaccharide in the group consisting of gellan, welan, S-88, S-198 and rhamsan gum, with the hydrogel being swellable to apply a force to the tissue after the introduction into the patient.

2. The device of claim 1, wherein the polysaccharide comprises a borate ester.

3. The device of claim 1, wherein the polysaccharide comprises an acidic polysaccharide depolymerized to lower the molecular weight of the acidic polysaccharide.

4. The device of claim 1, wherein the polysaccharide further comprises a salt.

5. The device of claim 5, wherein the salt comprises silver.

6. The device of claim 1, wherein the hydrogel is dehydrated before introduction into a patient and the predetermined structure comprises dehydrated particles made of the hydrogel.

7. The device of claim 1, wherein the polysaccharide molecules are substantially parallel to each other.

8. The device of claim 1 further comprising a therapeutic agent.

9. The device of claim 1 further comprising a member of the group consisting of a preservative agent, an antimicrobial agent, an agent that is both a preservative and an antimicrobial, or a combination thereof.

10. The device of claim 1, wherein the device is essentially completely degradable in less than about 7 days in vitro in a physiological saline solution kept at 37° C.

11. The device of claim 1, wherein the device is removable from a patient by exposing the polysaccharide to substantially deionized water.

12. The device of claim 1, wherein the plug is removable from a patient by exposing the device in the patient to a solution that is hypertonic relative to the device.

13. The device of claim 1, wherein the polysaccharide further comprises a metal and the polysaccharide is removable from a patient by oxidation catalyzed by the metal upon exposure to oxidizing agents.

14. The device of claim 1, wherein the predetermined structure is adapted to use with a treatment that is a member of the group consisting of abdominal aortic aneurysm, thoracic aortic aneurysms, chemoembolotherapy, tissue augmentation, replacement material for synovial fluid, adhesion prevention, large wound tamponade, and nasal or sinus cavity packing.

15. A method of occluding a lumen or void defined by a tissue in a patient comprising: introducing into the lumen or void a swellable medical device having a predetermined structure that comprises a biocompatible hydrogel comprising at least one polysaccharide in the group consisting of gellan, welan, S-88, S-198 and rhamsan, wherein the medical device swells after introduction to apply a force against the tissue that defines the lumen or void.

16. The method of claim 15, wherein the polysaccharide is processed from a solution of acidified polymer dissolved in an organic solvent.

17. The method of claim 15, wherein the polysaccharide further comprises a salt.

18. The method of claim 17, wherein the salt comprises silver, with the salt being formed by precipitatation or reduction upon contact with a suitable coagulation bath.

19. The method of claim 15, wherein the polysaccharide comprises a borate ester.

20. The method of claim 15, wherein the polysaccharide comprises an acidic polysaccharide that has been depolymerized to lower the molecular weight of the acidic polysaccharide.

21. The method of claim 15, wherein the hydrogel is dehydrated before introduction into the patient and the predetermined structure comprises dehydrated particles made of the hydrogel.

22. The method of claim 15, wherein the polysaccharide molecules are processed to make the molecules substantially parallel to each other.

23. The method of claim 15, further comprising introducing a therapeutic agent into the polysaccharide.

24. The method of claim 15, wherein the device is essentially completely degradable in less than about 7 days in vitro in a physiological saline solution kept at 37° C.

25. The method of claim 15, wherein the device is removable from a patient by exposing the polysaccharide to substantially deionized water.

26. The method of claim 15, wherein the plug is removable from a patient by exposing the device in the patient to a solution that is hypertonic relative to the device.

27. The method of claim 15, wherein the polysaccharide further comprises a metal and the polysaccharide is removable from a patient by oxidation catalyzed by the metal upon exposure to oxidizing agents.

28. The method of claim 15, wherein the lumen or void is associated with a treatment that is a member of the group consisting of abdominal aortic aneurysm, thoracic aortic aneurysms, chemoembolotherapy, tissue augmentation, replacement material for synovial fluid, adhesion prevention, large wound tamponade, and nasal or sinus cavity packing.

29. A biocompatible anisotropically swellable implant that is implantable into a tissue of a patient, the implant comprising a biocompatible material that anisotropically swells in vitro in a physiological saline solution when not subjected to constraining forces, with the material being anisotropically swellable in response to exposure to a physiological fluid upon introduction into the tissue to apply a force against the tissue.

30. The implant of claim 29, wherein the anisotropically swellable material comprises a volume, a first length and a second length perpendicular to the first length, wherein exposure to physiological fluid causes the volume to increase, the first length to undergo a first percentage increase and the second length to undergo a second percentage increase that is less than the first percentage increase for the first length.

31. The implant of clam 30, wherein the first percentage increase is at least 100%.

32. The implant of clam 30, wherein the second percentage increase is less than 0%.

33. The implant of clam 30, wherein the first length is structured to swell against the tissue, wherein the tissue is a portion of a duct, passage, orifice, or wound.

34. The implant of clam 29, wherein the material comprises polymers processed into an arrangement of polymers that are substantially parallel to each other.

35. The implant of clam 29, wherein the material comprises a polysaccharide.

36. The implant of clam 29, wherein the material comprises at least one member of the group consisting of gellan, welan, S-88, S-198, and a rhamsan gum.

37. The implant of clam 29, wherein the material comprises an acidic polysaccharide or salt thereof depolymerized to lower the molecular weight of the acidic polysaccharide.

38. The implant of clam 29 further comprising a therapeutic agent in the material.

39. The implant of clam 29 further comprising a preservative/antimicrobial agent in the material.

40. The implant of clam 29, wherein the device is removable by metal-catalyzed oxidation.

41. The implant of clam 29, wherein the device is removable by shrinkage upon exposure to a solution that is hypertonic relative to the material.

42. A method of occluding a lumen or void defined by a tissue in a patient, the method comprising implanting a device into the tissue that comprises a biocompatible material that anisotropically swells in vitro in a physiological saline solution when not subjected to constraining forces, with the material being anisotropically swellable in response to exposure to a physiological fluid upon introduction into the tissue to apply a force against the tissue.

43. The method of claim 42, wherein the anisotropically swellable material comprises a volume, a first length and a second length perpendicular to the first length, wherein exposure to physiological fluid causes the volume to increase, the first length to undergo a first percentage increase and the second length to undergo a second percentage increase that is less than the first percentage increase for the first length.

44. The method of claim 43, wherein the first percentage increase is at least 100% and the second percentage increase is less than 0%.

45. The method of claim 43, wherein the first length is structured to swell against the tissue, wherein the tissue is a portion of a duct, passage, orifice, or wound.

46. The method of claim 42, wherein the material comprises a polysaccharide.

47. The method of claim 42, wherein the material comprises at least one member of the group consisting of gellan, welan, S-88, S-198, and a rhamsan gum.

48. The method of claim 42 further comprising a therapeutic agent in the material.

49. The method of claim 42, further comprising removing the device following shrinkage of the device upon exposure to a solution that is hypertonic relative to the material.

50. A method of making an anisotropically swellable material from polymers comprising: aligning polymers in a substantially parallel orientation relative to each other to form the material, with the material being anisotropically swellable in a physiological solution.

51. The method of claim 50, wherein aligning the polymers comprises at least one technique chosen from the group consisting of spin coating, spray coating, stretching, unidirectional freezing, extrusion from liquid crystalline solution, ordered convection, and stretching plus drying of an extrusion.

52. The method of claim 50, wherein aligning the polymers comprises stretching the material.

53. The method of 50 further comprising soaking the polymeric material in a fluid comprising mineral acids, organic acids or salts of monovalent cations before stretching the polymeric material.

54. The method of claim 50, wherein the polymeric material comprises at least one member of the group consisting of gellan gum, welan, S-88, S-198, rhamsan gum, carboxymethylcellulose, alginic acid and salts thereof.

55. The method of claim 50, wherein aligning the polymers comprises acidification of anionic polymers or their conversion to salts of monovalent cations before dissolution in an organic solvent.

56. A medical device comprising: a hydrogel having a predetermined structure and being comprised of anionic polymers crosslinked by an insoluble metal salt.

57. The device of claim 56, wherein the anionic polymers comprise a polysaccharide.

58. The device of claim 56, wherein the anionic polymer comprises gellan, alginate, poly(acrylic acid), xanthan, carrageenan, carboxymethyl cellulose, carboxymethyl chitosan, hydroxypropyl carboxymethyl cellulose, pectin, welan, gum Arabic, karaya gum, psyllium seed gum, carboxymethyl guar, mesquite gum, or a combination thereof.

59. The device of claim 56, wherein the metal salt is formed from a metal with a valence of at least +2.

60. The device of claim 56, wherein the metal salt comprises a reaction product of a metal and a member of the group consisting of silicates, sulfides, halides, oxides, borates, carbonates, sulfates, phosphates, arsenates, vanadates, tungstates, molybdates, hydroxides, and chromates.

61. The device of claim 56, wherein the hydrogel is swellable by at least 100% in volume after exposure to physiological fluids in a patient.

62. The device of claim 56 further comprising a therapeutic agent.

63. The device of claim 56, wherein the predetermined structure is introducible into a lumen or void associated with a treatment that is a member of the group consisting of abdominal aortic aneurysm, thoracic aortic aneurysms, chemoembolotherapy, tissue augmentation, replacement material for synovial fluid, adhesion prevention, large wound tamponade, and nasal or sinus cavity packing.

64. The device of claim 56, further comprising unmineralized free metal ion-binding functional groups.

65. The device of claim 56, wherein the hydrogel further comprises covalent crosslinks.

66. The device of claim 56, wherein the hydrogel comprises polymers crosslinked by a reaction of hydroxyl groups on the polymers with crosslinking agents.

67. The device of claim 56, wherein the hydrogel is degradable by metal-catalyzed oxidation.

68. A method of removing a biocompatible hydrogel from a patient by an oxidative-reductive reaction, comprising: exposing the hydrogel in the patient to a metal ion catalyst that bonds to the hydrogel and catalyzes the oxidation of the hydrogel upon exposure to an oxidizing agent to degrade the hydrogel.

69. The method of claim 68 further comprising making the hydrogel, wherein the hydrogel has functional groups for binding the metal ion catalyst.

70. The method of claim 68, wherein the metal ions comprise heavy metal, transition metal, ferrous, ferric, cuprous, or cupric ions.

71. The method of claim 68, wherein the oxidizing agents comprise a phenol, phenolic compound, benzoyl peroxide, hydrogen peroxide, or ascorbate.

72. A method of removing a medical device from a patent comprising exposing the device to substantially deionized water to dissolve the device, wherein the device comprises a biocompatible hydrogel comprising at least one polysaccharide in the group consisting of gellan, welan, S-88, S-198 and rhamsan.

73. The method of claim 72, wherein the device comprises a shaft and a head at a proximal end of the shaft, with the shaft comprising an introducible portion for introduction into a patient.

74. The method of claim 72, wherein the polysaccharide comprises an acidic polysaccharide or salt thereof depolymerized to lower the molecular weight of the acidic polysaccharide.

75. The method of claim 72 further comprising copper or iron associated with the polysaccharide.

76. The method of claim 72, wherein the polysaccharide comprises polymers processed into an arrangement of polymers that are substantially parallel to each other.

77. The method of claim 72, with the device further comprising a therapeutic agent.

78. The method of claim 72, wherein the device further comprises a metal and the plug is degradable by metal-catalyzed oxidation using an oxidation agent.

79. The method of claim 72 further comprising shrinking the hydrogel by exposure to a solution hypertonic relative to the hydrogel.

80. A method of shrinking a biocompatible hydrogel in a patient, the method comprising exposing the biocompatible hydrogel in the patient to a solution that is hypertonic relative to the device.

81. The method of claim 80, wherein the device comprises a shaft and a head at a proximal end of the shaft, with the shaft comprising an introducible portion for introduction into a patient.

82. The method of claim 80, wherein the polysaccharide comprises an acidic polysaccharide or salt thereof depolymerized to lower the molecular weight of the acidic polysaccharide.

83. The method of claim 80 further comprising copper or iron associated with the polysaccharide.

84. The method of claim 80, wherein the polysaccharide comprises polymers processed into an arrangement of polymers that are substantially parallel to each other.

85. The method of claim 80, with the device further comprising a therapeutic agent.

86. The method of claim 80, wherein the device further comprises a metal and the plug is degradable by metal-catalyzed oxidation using an oxidation agent.
